SEQUENCE LISTING (38 sequences)

SEQ ID NO: 1 - protein, 155 aa, Homo sapiens
  NOTE (1)..(155): FGF1 WT (1-155aa)
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 2 - protein, 136 aa, Artificial Sequence
  FGF1 (22-155aa) - FGF1(delta155aa) - FGF_deltaWT
MANYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 3 - protein, 155 aa, Artificial Sequence
  FGF1(155aa;S114A) (M1)
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYIAKKHAEKNWFVGLKKNGSCKRGPRTHYGQKAILFLPLPVSSD

SEQ ID NO: 4 - protein, 155 aa, Artificial Sequence
  FGF1(155aa;L150D) (M2)
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPDPVSSD

SEQ ID NO: 5 - protein, 155 aa, Artificial Sequence
  FGF1(155aa;S114A/L150D) (M3)
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYIAKKHAEKNWFVGLKKNGSCKRGPRTHYGQKAILFLPDPVSSD

SEQ ID NO: 6 - protein, 155 aa, Artificial Sequence
  FGF1(155aa;Q55P) (M4)
MAEGEITTFTALTEKFNLPPGNYKKPKLLYCSNGGHFLRILPDGTVDGTRDRSDPHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 7 - protein, 155 aa, Artificial Sequence
  FGF1(155aa;S62I) (M5)
MAEGEITTFTALTEKFNLPPGNYKKPKLLYCSNGGHFLRILPDGTVDGTRDRSDQHIQLQLIAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 8 - protein, 155 aa, Artificial Sequence
  FGF1(155aa;H108G) (M6)
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENGYNTYISKKHAEKNWFVGLKKNGSCKRGPRTHYGQKAILFLPLPVSSD

SEQ ID NO: 9 - protein, 155 aa, Artificial Sequence
  FGF1(155aa;Q55P/S62I/H108G) (M7)
MAEGEITTFTALTEKFNLPPGNYKKPKLLYCSNGGHFLRILPDGTVDGTRDRSDPHIQLQLIAESVGEVYIKSTETGQYLAMDTDGLLYGSQTPNEECLFLERLEENGYNTYISKKHAEKNWFVGLKKNGSCKRGPRTHYGQKAILFLPLPVSSD

SEQ ID NO: 10 - protein, 155 aa, Artificial Sequence
  FGF1(155aa;Q55P/S62I/H108G/S114A/L150D) (M8)
MAEGEITTFTALTEKFNLPPGNYKKPKLLYCSNGGHFLRILPDGTVDGTRDRSDPHIQLQLIAESVGEVYIKSTETGQYLAMDTDGLLYGSQTPNEECLFLERLEENGYNTYIAKKHAEKNWFVGLKKNGSCKRGPRTHYGQKAILFLPDPVSSD

SEQ ID NO: 11 - protein, 136 aa, Artificial Sequence
  FGF1(delta155aa;S114A) (M9)
MANYKKPKLLYCSNGGHFLRILPDGTVDGTRDRSDQHIQLQLSAESVGEVYIKSTETGQYLAMDTDGLLYGSQTPNEECLFLERLEENHYNTYIAKKHAEKNWFVGLKKNGSCKRGPRTHYGQKAILFLPLPVSSD

SEQ ID NO: 12 - protein, 136 aa, Artificial Sequence
  FGF1(delta155AA;L150D) (M10)
MANYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPDPVSSD

SEQ ID NO: 13 - protein, 136 aa, Artificial Sequence
  FGF1(delta155aa;S114A/L150D) (M11)
MANYKKPKLLYCSNGGHFLRILPDGTVDGTRDRSDQHIQLQLSAESVGEVYIKSTETGQYLAMDTDGLLYGSQTPNEECLFLERLEENHYNTYIAKKHAEKNWFVGLKKNGSCKRGPRTHYGQKAILFLPDPVSSD

SEQ ID NO: 14 - protein, 136 aa, Artificial Sequence
  FGF1(delta155aa;Q55P) (M12)
MANYKKPKLLYCSNGGHFLRILPDGTVDGTRDRSDPHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 15 - protein, 136 aa, Artificial Sequence
  FGF1(delta155aa;S62I) (M13)
MANYKKPKLLYCSNGGHFLRILPDGTVDGTRDRSDQHIQLQLIAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 16 - protein, 136 aa, Artificial Sequence
  FGF1(delta155aa;H108G) (M14)
MANYKKPKLLYCSNGGHFLRILPDGTVDGTRDRSDQHIQLQLSAESVGEVYIKSTETGQYLAMDTDGLLYGSQTPNEECLFLERLEENGYNTYISKKHAEKNWFVGLKKNGSCKRGPRTHYGQKAILFLPLPVSSD

SEQ ID NO: 17 - protein, 136 aa, Artificial Sequence
  FGF1(delta155aa;Q55P/S62I/H108G) (M15)
MANYKKPKLLYCSNGGHFLRILPDGTVDGTRDRSDPHIQLQLIAESVGEVYIKSTETGQYLAMDTDGLLYGSQTPNEECLFLERLEENGYNTYISKKHAEKNWFVGLKKNGSCKRGPRTHYGQKAILFLPLPVSSD

SEQ ID NO: 18 - protein, 136 aa, Artificial Sequence
  FGF1(delta155aa;Q55P/S62I/H108G/S114A/L150D) (M16)
MANYKKPKLLYCSNGGHFLRILPDGTVDGTRDRSDPHIQLQLIAESVGEVYIKSTETGQYLAMDTDGLLYGSQTPNEECLFLERLEENGYNTYIAKKHAEKNWFVGLKKNGSCKRGPRTHYGQKAILFLPDPVSSD

SEQ ID NO: 19 - protein, 322 aa, Artificial Sequence
  FGF1_DIMER(155aa) (FGF1_WT_DIMER)
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSDGGGGSGGGGSGGGGAEGEITTFTALTEKFNLPPNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 20 - protein, 285 aa, Artificial Sequence
  FGF1_DIMER(delta155aa) (FGF1_deltaWT_DIMER)
MANYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSDGGGGSGGGGSGGGGANYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 21 - protein, 322 aa, Artificial Sequence
  FGF1_M8_DIMER(155aa)
MAEGEITTFTALTEKFNLPPGNYKKPKLLYCSNGGHFLRILPDGTVDGTRDRSDPHIQLQLIAESVGEVYIKSTETGQYLAMDTDGLLYGSQTPNEECLFLERLEENGYNTYIAKKHAEKNWFVGLKKNGSCKRGPRTHYGQKAILFLPDPVSSDGGGGSGGGGSGGGGAEGEITTFTALTEKFNLPPNYKKPKLLYCSNGGHFLRILPDGTVDGTRDRSDPHIQLQLIAESVGEVYIKSTETGQYLAMDTDGLLYGSQTPNEECLFLERLEENGYNTYIAKKHAEKNWFVGLKKNGSCKRGPRTHYGQKAILFLPDPVSSD

SEQ ID NO: 22 - protein, 285 aa, Artificial Sequence
  FGF1_M16_DIMER(delta155aa)
MANYKKPKLLYCSNGGHFLRILPDGTVDGTRDRSDPHIQLQLIAESVGEVYIKSTETGQYLAMDTDGLLYGSQTPNEECLFLERLEENGYNTYIAKKHAEKNWFVGLKKNGSCKRGPRTHYGQKAILFLPDPVSSDGGGGSGGGGSGGGGANYKKPKLLYCSNGGHFLRILPDGTVDGTRDRSDPHIQLQLIAESVGEVYIKSTETGQYLAMDTDGLLYGSQTPNEECLFLERLEENGYNTYIAKKHAEKNWFVGLKKNGSCKRGPRTHYGQKAILFLPDPVSSD

SEQ ID NO: 23 - protein, 155 aa, Artificial Sequence
  FGF1_M(155aa)
MAEGEITTFTALTEKFNLPPGNYKKPKLLYCSNGGHFLRILPDGTVDGTRDRSDPHIQLQLIAESVGEVYIKSTETGQYLAMDTDGLLYGSQTPNEECLFLERLEENGYNTYISKKHAEKNWFVGLKKNGSCKRGPRTHYGQKAILFLPLPVASD

SEQ ID NO: 24 - protein, 136 aa, Artificial Sequence
  FGF1_M(delta155aa)
MANYKKPKLLYCSNGGHFLRILPDGTVDGTRDRSDPHIQLQLIAESVGEVYIKSTETGQYLAMDTDGLLYGSQTPNEECLFLERLEENGYNTYISKKHAEKNWFVGLKKNGSCKRGPRTHYGQKAILFLPLPVASD

SEQ ID NO: 25 - protein, 155 aa, Artificial Sequence
  FGF1_M(155aa)
MAEGEITTFTALTEKFNLPPGNYKKPKLLYCSNGGHFLRILPDGTVDGTRDRSDPHIQLQLIAESVGEVYIKSTETGQYLAMDTDGLLYGSQTPNEECLFLERLEENGYNTYISKKHAEKNWFVGLKKNGSCKRGPRTHYGQKAILFLPLPVRSD

SEQ ID NO: 26 - protein, 136 aa, Artificial Sequence
  FGF1_M(delta155aa)
MANYKKPKLLYCSNGGHFLRILPDGTVDGTRDRSDPHIQLQLIAESVGEVYIKSTETGQYLAMDTDGLLYGSQTPNEECLFLERLEENGYNTYISKKHAEKNWFVGLKKNGSCKRGPRTHYGQKAILFLPLPVRSD

SEQ ID NO: 27 - protein, 155 aa, Artificial Sequence
  FGF1_M(155aa)
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVDDD

SEQ ID NO: 28 - protein, 136 aa, Artificial Sequence
  FGF1_M(delta155aa)
MANYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVDDD

SEQ ID NO: 29 - protein, 155 aa, Artificial Sequence; identical to SEQ ID NO: 27
  FGF1_M(155aa)

SEQ ID NO: 30 - protein, 136 aa, Artificial Sequence
  FGF1_M(delta155aa)
MANYKKPKLLYCSNGGHFLRILPDGTVDGTRDRSDPHIQLQLIAESVGEVYIKSTETGQYLAMDTDGLLYGSQTPNEECLFLERLEENGYNTYISKKHAEKNWFVGLKKNGSCKRGPRTHYGQKAILFLPLPVDDD

SEQ ID NO: 31 - protein, 155 aa, Artificial Sequence
  FGF1 (1-155aa) FGF1_M(155aa)
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVRSD

SEQ ID NO: 32 - protein, 155 aa, Artificial Sequence
  FGF1 (22-155aa) FGF1_M(delta155aa)
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVASD

SEQ ID NO: 33 - protein, 136 aa, Artificial Sequence
  FGF1 (22-155aa) FGF1_M(delta155aa)
MANYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVRSD

SEQ ID NO: 34 - protein, 136 aa, Artificial Sequence
  FGF1 (22-155aa) FGF1_M(delta155aa)
MANYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVASD

SEQ ID NO: 35 - protein, 322 aa, Artificial Sequence
  FGF1 (1-155aa) FGF1_DIMER(155aa)
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVRSDGGGGSGGGGSGGGGAEGEITTFTALTEKFNLPPNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVRSD

SEQ ID NO: 36 - protein, 322 aa, Artificial Sequence
  FGF1 (1-155aa) FGF1_DIMER(155aa)
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVASDGGGGSGGGGSGGGGAEGEITTFTALTEKFNLPPNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVASD

SEQ ID NO: 37 - protein, 285 aa, Artificial Sequence
  FGF1 (22-155aa) FGF1_DIMER(delta155aa)
MANYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVRSDGGGGSGGGGSGGGGANYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVRSD

SEQ ID NO: 38 - protein, 285 aa, Artificial Sequence
  FGF1 (22-155aa) FGF1_DIMER(delta155aa)
MANYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVASDGGGGSGGGGSGGGGANYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVASD

(19) United States
(12) Patent Application Publication
(10) Pub. No.: US 2025/0282841 A1
(43) Pub. Date: Sep. 11, 2025

(54) HUMAN FIBROBLAST GROWTH FACTOR 1 (FGF-1) MUTEINS, THEIR DIMERS AND USES

(71) Applicants: Celon Pharma S.A., Kielpin/Lomianki (PL); Uniwersytet Wroclawski, Wroclaw (PL)

(72) Inventors: Jacek Józef OTLEWSKI, Wroclaw (PL); Malgorzata Urszula ZAKRZEWSKA, Wroclaw (PL); Daniel Rafal KROWARSCH, Wroclaw (PL); Aleksandra Anna CZYREK, Wroclaw (PL); Michal Mieczyslaw JANISZEWSKI, Tarnów (PL); Jerzy Szczepan PIECZYKOLAN, Warszawa (PL); Ewa DRZAZGA, Konskie (PL); Katarzyna BAZYDLO-GUZENDA, Warszawa (PL); Pawel BUDA, Warszawa (PL); Maciej WIECZOREK, Kielpin/Lomianki (PL)

(73) Assignees: Celon Pharma S.A., Kielpin/Lomianki (PL); Uniwersytet Wroclawski, Wroclaw (PL)

(21) Appl. No.: 18/564,350

(22) PCT Filed: May 25, 2022
(86) PCT No.: PCT/PL2022/050032

§ 371 Date: Nov. 27, 2023

(30) Foreign Application Priority Data
May 26, 2021 (PL) P.438001

(51) Int. Cl. C07K 14/50; A61K 38/00; A61P 3/10

(52) U.S. Cl. CPC C07K 14/501; A61P 3/10; A61K 38/00

(57) ABSTRACT

The present invention relates to human fibroblast growth factor (FGF-1) muteins having decreased mitogenicity, dimers of human FGF-1 muteins, as well as such human FGF-1 muteins and dimers of such muteins for use in reducing blood glucose level, particularly for use in the treatment of diabetes.

### FIELD OF THE INVENTION

The invention relates to human fibroblast growth factor 1 (FGF-1) muteins having decreased mitogenicity, dimers of such FGF-1 muteins having decreased mitogenicity (both homodimers and heterodimers of such muteins) having the ability to reduce glucose level after administration, and uses thereof in medicine, pharmacy and in therapy, in particular in the treatment of diabetes, including type 2 diabetes (T2D).

### BACKGROUND ART

Disorders of metabolic homeostasis are manifested inter alia in obesity and resistance of cells, in particular resistance of peripheral cells to the action of insulin (insulin resistance). This results in weaker glucose absorption from the blood and consequently the development of type 2 diabetes. Diabetes is currently classified as one of the most serious civilization diseases in the world. The number of subjects suffering from diabetes continues to increase and the complications that accompany it pose a threat to the health and life of patients. The main symptom of diabetes is a high blood glucose level (hyperglycemia). If such condition persists for a long time, it causes injury to blood vessels and arteries. This leads to blood supply disorders and dysfunction of many organs and consequently to chronic complications: diabetic retinopathy, diabetic nephropathy and diabetic neuropathy. Diabetes also accelerates the development of atherosclerotic lesions in blood vessels leading to the development of ischaemic heart disease and lower extremities, as well as cerebral stroke. Despite advances in medicine, currently used therapies still have a number of undesirable side effects, such as the risk of occurrence of too low blood glucose level (hypoglycaemia), fatty liver or bone mass loss. Due to the observed continuous increase in the number of patients with T2D, new therapies directed at eliminating insulin resistance are being sought. Among the most effective medicaments currently used are thiazolidinediones (TZDs), which are antagonists of nuclear PPARγ receptors. These receptors regulate adipogenesis, lipid metabolism and stimulate insulin mediators that mediate glucose uptake by peripheral tissues. Despite their high effectiveness, the use of TZDs involves a number of side effects, such as weight gain, bone mass loss or heart failure. That is why other mediators of PPAR receptors are being sought, which will continue to be characterized by a high insulin sensitizing potential but will not entail a risk of side effects. In the treatment of T2D combined therapies are also used, i.e. simultaneous administration of several medicaments, because the majority of patients do not respond to single medicaments or quickly become insensitive thereto. Despite advancements in medicine and the introduction of new medicaments, it is still impossible to fully control the disease and its complications. One important aspect in controlling metabolic homeostasis is the ability of adipose tissue cells to remodel and alter basal metabolism in response to fluctuations in the availability of nutrients. In 2012, Jonker et al. presented studies that were aimed at identifying diet-controlled genes in various tissues involved in metabolism (muscles, liver, brown adipose tissue (BAT), white adipose tissue (WAT)). It was proven that FGF-1 is selectively induced in visceral (gonadal) white adipose tissue (gWAT) in response to a diet with high content of fat (Jonker, J. W., Suh, J. M., Atkins, A. R., Ahmadian, M., Li, P., Whyte, J., He, M., Juguilon, H., Yin, Y., Phillips, C. T., Yu, R. T., Olefsky, J. M., Henry, R. R., Downes, M., & Evans, R. M. (2012). A PPARγ-FGF1 axis is required for adaptive adipose remodelling and metabolic homeostasis. Nature, 485(7398), 391-394. https://doi.org/10.1038/nature10998). Subsequent in vivo experiments proved that deletion of FGF-1 gene in mice leads to a systemic metabolic dysfunction and an increase in insulin resistance. Mice without FGF-1 gene that were on high-fat diet developed an aggressive diabetic phenotype resulting from a limited growth of adipose tissue. Additionally, the authors demonstrated that induction of FGF-1 in WAT is controlled by nuclear PPARγ receptors (Jonker et al., 2012).

The family of fibroblast growth factors FGF in humans and in rodents consists of 22 genes encoding structurally related polypeptides. These genes are located in various chromosomes, which indicates that the FGF family was formed as a result of duplication and translocation of genes and chromosomes. FGF-1 protein is a paracrine protein, which is secreted by various types of cells (e.g. fibroblasts, adipocytes) and acts locally. Human FGF-1 (hFGF1) consists of 155 amino acids (aa), of which the first 14 aa constitute a propeptide, which is removed during maturation in the process of cellular protein expression. In the literature there are known recombinant human (rh) forms of FGF-1 having three lengths: full length (155 aa) and two N-terminal deletion forms (including amino acids 15-155 aa and 21-155 aa). According to the present knowledge, all three forms have a similar binding to FGFRs and heparin, and similar mitogenicity. FGF-1 is a known and strong mitogen for many cells (it stimulates cell division and proliferation). FGF-1 ligand binds to all four cellular FGF receptors (FGFR1-4). A protein-receptor complex is stabilized by heparans, polysaccharides naturally present on cell surface. Literature indicates that a native (in a naturally occurring state, wild-type) FGF-1 protein is characterized by low thermal stability (the denaturation temperature is about 40° C.), and thus a short biological half-life (the half-life in DMEM is about 1.1-2 hours) (Zakrzewska, M., Krowarsch, D., Wiedlocha, A., & Otlewski, J. (2004). Design of fully active FGF-1 variants with increased stability. Protein Engineering Design and Selection, 17(8), 603-611. https://pubmed.ncbi.nlm.nih.gov/16126225/).

FGF-1 protein endogenously produced by an organism is secreted inter alia in adipose tissue and is necessary to maintain the metabolic homeostasis of this tissue. Loss of the ability to synthesize FGF-1 results in the development of T2D. Current literature indicates studies the purpose of which was to determine the effect of exogenous administration of FGF-1 protein (more specifically, a recombinant human FGF-1: rhFGF-1) to animals with type 2 diabetes induced by eating a high-fat diet. The purpose of those studies was to determine what effect the administration of FGF-1 ligand had on reducing the blood glucose level in animals and restoring metabolic homeostasis. It was proved that rhFGF-1 lowers blood glucose concentration. Moreover, it was proved that rhFGF-1 reduces glucose concentration while not causing hypoglycaemia, which is a considerable advantage over the medicaments currently used in the treatment of type 2 diabetes. However, due to strong mitogenic properties and short half-life (quick degradation) rhFGF-1 cannot be administered to patients with type 2 diabetes. Strong mitogenicity of FGF-1 may cause many negative effects including induction of neoplasia. Nevertheless, due to the advantageous mechanism of action consisting in sensitizing cells to the action of endogenous insulin, and no risk of hypoglycaemia, rhFGF-1 is an interesting candidate for further therapeutic development in the treatment of T2D.

In 2014, Suh at al demonstrated the antidiabetic effect of subcutaneously administered FGF-1 in animal models of diabetes induced by eating high-fat diet (Suh, J. M., Jonker, J. W., Ahmadian, M., Goetz, R., Lackey, D., Osborn, O., Huang, Z., Liu, W., Yoshihara, E., van Dijk, T. H., Havinga, R., Fan, W., Yin, Y.-Q., Yu, R. T., Liddle, C., Atkins, A. R., Olefsky, J. M., Mohammadi, M., Downes, M., & Evans, R. M. (2014). Endocrinization of FGF1 produces a neomorphic and potent insulin sensitizer. Nature, 513(7518), 436-439. https://doi.org/10.1038/nature13540). In their experiments Suh et al. used the full-length (155 aa) rhFGF1 protein. Table 1 contains information concerning the study models used by Suh et al.

TABLE 1
List and brief characteristics of animals models of
diabetes used in experiments by Suh et al (SALK
Institute) (King, A. J. F. (2012). The use of animal
models in diabetes research. British Journal of
Pharmacology, 166(3), 877-894.
https://doi.org/10.1111/j.1476-5381.2012.01911.x).

| | | Most important phenotype |
|---|---|---|
| Animal model | Type of change | features |
| ob/ob | Mutation of leptin | Obesity |
| | protein | Hyperglycaemia |
| | | Insulin resistance |
| | | Pancreatic β-cells secrete |
| | | insulin(Type 2 diabetes) |
| db/db | Mutation of leptin | Obesity |
| | receptor | Hyperglycaemia |
| | | Insulin resistance |
| | | Pancreatic β-cells secrete |
| | | insulin(Type 2 diabetes) |
| DIO (Diet | High-fat diet | Obesity |
| Induced Obesity) | | Hyperglycaemia |
| | | Pancreatic β-cells secrete |
| | | insulin(Type 2 diabetes) |
| STZ | Administration of | Lack of obesity |
| | streptozotocin | Hyperglycaemia |
| | | Damaged pancreatic |
| | | β-cells do not secrete |
| | | insulin (Type 1 diabetes) |
| C57BL/6J | Mice fed with | Healthy animals |
| | standard feed | |

In all animal groups representing type 2 diabetes phenotype a significant reduction in blood glucose concentrations was observed after administration of rhFGF-1. As expected, in the case of the STZ model (mice characterized by lack of insulin secretion by pancreatic B-cells), which represents Type 1 diabetes phenotype, rhFGF-1 did not cause reduction of blood glucose. This result allows one to conclude that this protein does not mimic the biological effect achieved by administration of insulin. In the case of healthy animals (with the physiological value of blood glucose concentration of about 135 mg/dl), administration of the protein was neutral and did not reduce the blood glucose concentration. The antidiabetic effect of a single administration of rhFGF-1 was maintained for at least subsequent 24 hours. The action of rhFGF-1 proved to be dose-dependent; however, the hypoglycaemic effect was not observed for any dose used. Contrary to thiazolidinediones, rhFGF-1 did not cause weight gain and did not decrease bone density, but it contributed to reduction of hepatic steatosis level (TZDs increase it), which was fact was observed during rhFGF-21 therapy.

Below is presented the used by Suh et al. sequence of a full-length human recombinant rhFGF-1 (155 aa):

MAEGEITTFTALTEKFNLPPGNYKKPKLLYCSNGGHFLRILPDGTV
DGTRDRSDQHIQLQLSAESVGEVYIKSTETGQYLAMDTDGLLYGSQ
TPNEECLFLERLEENHYNTYISKKHAEKNWFVGLKKNGSCKRGPRT
HYGQKAILFLPLPVSSD

As indicated above, the native FGF-1 protein is characterized by a high mitogenic potential, which leads to excessive proliferation of cells and involves a risk of neoplasia. It is known that FGF-1 exhibits strong affinity to its cellular receptors (FGFR), which results in a strong intracellular signal transduction. FGF-1 ligand binding to the receptor triggers an intracellular signalling cascade inducing intensive cell divisions. Suh et al. verified whether it was possible to separate the mitogenic potential of FGF-1 from its antidiabetic activity. For this purpose they developed an FGF-1 mutant lacking 24 N-terminal amino acids. They assumed that removal of those amino acids would reduce the strength of the FGF-1 ligand binding to the FGFR receptor, which would result in decreased mitogenicity of the protein.

The sequence of the developed recombinant human truncated (shortened) by 24 aa FGF-1 (FGF1^{ΔNT}; K25-D155) is as follows:

MKPKLLYCSNGGHFLRILPDGTVDGTRDRSDQHIQLQLSAESVGEV
YIKSTETGQYLAMDTDGLLYGSQTPNEECLFLERLEENHYNTYISK
KHAEKNWFVGLKKNGSCKRGPRTHYGQKAILFLPLPVSSD

As predicted, the truncated ligand showed a lower affinity to FGFRs, which resulted in a significant reduction of mitogenic effect, however FGF1^{ΔNT} retained the antidiabetic potential observed for the wild-type protein.

In order to learn the mechanism of action of FGF-1 and the role of the basal receptor binding FGF-1 ligand (FGFR), Suh et al. developed another mutant (FGF1^{ΔNT2}), from which the first 28 N-terminal amino acids were removed (i.e. amino acids: MAEGEITTFT ALTEKFNLPP GNYKKPKL). Such a change blocked the binding with FGFR1 and deprived the protein of its antidiabetic properties.

The initial sequence of the used for modification rhFGF-1 was as follows:

MAEGEITTFTALTEKFNLPPGNYKKPKLLYCSNGGHFLRILPDGTV
DGTRDRSDQHIQLQLSAESVGEVYIKSTETGQYLAMDTDGLLYGSQ
TPNEECLFLERLEENHYNTYISKKHAEKNWFVGLKKNGSCKRGPRT
HYGQKAILFLPLPVSSD

(Amino acids that were omitted in the synthesis of FGF1^{ΔNT2} are marked with bold font.) The final sequence of the used human FGF-1 truncated by 28 aa (FGF-1^{ΔNT2}; L29-D155) obtained by Suh et al:

MLYCSNGGHFLRILPDGTVDGTRDRSDQHIQLQLSAESVGEVYIKS
TETGQYLAMDTDGLLYGSQTPNEECLFLEREENHYNTYISKKHAEK
NWFVGLKKNGSCKRGPRTHYGQKAILFLPLPVSSD

Table 2 below is a compilation of the most essential features of the native FGF-1 protein; however, due to the natural properties of this protein its therapeutic use in this form (unchanged) is impossible.

TABLE 2
Advantages resulting from the use of FGF-1 as a potential therapeutic and features
preventing the use of its native form in the treatment of Type 2 diabetes.

| Advantages | Comments | Disadvantages |
|---|---|---|
| Long lasting effect | According to literature, the effect of | Mitogenic properties of |
| | lower blood glucose concentration after | the protein, which may |
| | administration of FGF-1 remains for a | induce neoplasia in the |
| | minimum of subsequent 24 hours. The | long term. |
| | majority of medicaments currently | |
| | available on the market should be taken | |
| | at least twice a day. Such a long effect | |
| | of FGF-1 makes it possible to reduce the | |
| | number of times the medicament must | |
| | be taken, which translates into a better | |
| | comfort of patient's life. | |
| Mechanism of action | The action of the medicament is directed | Instability of the protein |
| advantageous for an | at sensitizing cells to the action of | and short half-life in vivo |
| organism | insulin. This means, when the | |
| | medicament is administered, the insulin | |
| | that is endogenously secreted by | |
| | pancreatic β-cells, will be more | |
| | effectively used by the organism. If the | |
| | organism is able to use its own insulin | |
| | resources, there will be no necessity to | |
| | use insulin preparations (exogenous | |
| | insulin) or the necessity to use | |
| | exogenous insulin will arise later and/or | |
| | smaller doses of insulin will be required. | |
| No risk of | All data from experiments and literature | |
| hypoglycaemia | univocally prove that FGF-1 does not | |
| | cause hypoglycaemia, which increases | |
| | safety of the therapy. | |

The studies of Suh et al. were among the first ones to seek modifications for therapeutic use of mutated FGF-1 ligand. At present, several strategies are known and have been described in the literature which are aimed at reducing mitogenic potential while preserving a positive biological effect in controlling metabolic homeostasis. These strategies encompass purposeful introduction of changes in the amino acids sequences of FGF-1 protein in various areas:
1. Deleting the first 24 aa (from N terminus) of the full-length hFGF-1 protein, and thus shortening (truncation of) the chain from 155 aa to 131 aa. This method of separating the antidiabetic potential from the mitogenic potential was used by Suh et al and was described in their study (Suh et al., 2014).
2. Producing a partial FGFR agonist, characterized by a mutation at the heparan sulphate binding site. FGF-1 requires heparan sulphate for binding with a receptor, dimerization and activation of the cellular path. Only a stable FGF-1:FGFR complex triggers a mitogenic response. Huang et al. developed a mutant FGF-1 protein with three local mutations at the heparan binding site (Huang, Z., Tan, Y., Gu, J., Liu, Y., Song, L., Niu, J., Zhao, L., Srinivasan, L., Lin, Q., Deng, J., Li, Y., Conklin, D. J., Neubert, T. A., Cai, L., Li, X., & Mohammadi, M. (2017). Uncoupling the Mitogenic and Metabolic Functions of FGF1 by Tuning FGF1-FGF Receptor Dimer Stability. Cell Reports, 20(7), 1717-1728. https://doi.org/10.1016/j.celrep.2017.06.063). Thereby, the effectiveness of FGF-1:FGFR:HS (heparan sulphate) complex formation was diminished and the mitogenic response of cells was significantly decreased in the studied conditions. The mutant is known in the literature as FGF-1^{ΔHES} and it has mutations at positions 127, 128, 133 of the polypeptide chain (Huang et al., 2017).
3. Reducing affinity to CK2 kinase. After exogenous administration of FGF-1, it binds to FGFR. The formation of FGF-1:FGFR1 complex results in the dimerization of receptors, and consequently phosphorylation of intracellular receptor domains. The complex is also internalized and FGF-1 is translocated into the nucleus where it stimulates DNA synthesis. Due to pleiotropy of biological effects caused by FGF-1 ligand a number of cytosolic and nuclear proteins were identified, which were responsible for direct interaction with the ligand. One of them is pleiotropic, constitutively active CK2 kinase consisting of two subunits a and two subunits B. Data from the literature indicate the fact that both subunits interact with FGF-1. The study of Skjerpen et al. demonstrates a correlation between the affinity to CK2 of a number of various FGF-1 mutants and their mitogenic potential (Skjerpen, C. S., Nilsen, T., Wesche, J., & Olsnes, S. (2002). Binding of FGF-1 variants to protein kinase CK2 correlates with mitogenicity. The EMBO Journal, 21(15), 4058-4069. https://doi.org/10.1093/emboj/cdf402). It was found out that mutations comprising the naturally occurring amino acid—lysine (L), which is replaced with a neutral or negatively charged amino acid lead to the reduction or lack of protein binding to CK2. Among the tested variants were proteins with lysine and serine(S) substitutions (K133R, K133A, K133E, S114A, S131E, S131E/K133E, substitutions counted relative to the full-length chain, 155 aa). Mutations S131A, S131A/K133A were characterized by a lower mitogenic potential compared to the wild-type. K133E mutation did not show a mitogenic potential (Skjerpen et al., 2002). These results are in line with the data obtained from other studies (K133E mutation is also present as one of three mutations in FGF-1^{ΔHBS} variant). Another problem related to the use of native human FGF-1 is its poor stability and a short biological half-life (Culajay, J. F., Blaber, S. I., Khurana, A., & Blaber, M. (2000). Thermodynamic characterization of mutants of human fibroblast growth factor 1 with an increased physiological half-life. Biochemistry, 39(24), 7153-7158. https://doi.org/10.1021/bi9927742). Poor stability is an essential limiting factor in the pharmacological applications of proteins, because it impedes the process of formulation or storage and use of biological medicaments. (Wang, W. (1999). Instability, stabilization, and formulation of liquid protein pharmaceuticals. In International Journal of Pharmaceutics (Vol. 185, Issue 2). https://doi.org/10.1016/S0378-5173(99)00152-0). Moreover, proteins in living organisms are exposed to various factors, such as non-optimal temperature, pH, oxygen free radicals or action of proteases. These factors may lead to protein unfolding, aggregation or proteolytic degradation. In the conditions of physiological pH and temperature of about 50% a protein is unfolded, because the denaturation temperature of FGF-1 is about 40° C. Thus, increasing the thermal stability of a protein would have a direct effect on its biological and antidiabetic activity. An increase in the thermal stability of a protein may be achieved by introducing point mutations into the polypeptide chain of the protein. Culajay et al. proved that a single H108G mutation in a truncated recombinant human rhFGF-1 protein increased its denaturation temperature by about 8° C. and thus increased its stability (Table 3 below) Culajay et al., 2000).

There have also been identified another two point mutations increasing the thermal stability of human FGF-1 (Q55P and S62I). They increased denaturation temperature by 8.1 and 9.3° C., respectively, relative to the native protein (Zakrzewska, Malgorzata, Krowarsch, D., Wiedlocha, A., Olsnes, S., & Otlewski, J. (2005). Highly Stable Mutants of Human Fibroblast Growth Factor-1 Exhibit Prolonged Biological Action. Journal of Molecular Biology, 352(4), 860-875. https://doi.org/10.1016/j.jmb.2005.07.066). The same team generated subsequent variants comprising multiple substitutions, including: Q55P/S62I and Q55P/S62I/H108G. The obtained results indicate that, among all studied mutations, it was the triple substitution that proved to stabilize a protein, increase denaturation temperature and prevent against proteolysis the most. (Table 3 below). The triple mutation also prolonged the protein half-life by nearly 10 hours (by comparison, the half-life of native FGF1 protein is about 1.5 h). It should be pointed out that the data concerning the half-lives of proteins concern preparations without heparin addition (heparin prevents wild-type FGF-1 protein from thermal and proteolytic inactivation) (Malgorzata Zakrzewska et al., 2005). Analyses showed that digestion of Q55P/S62I/H108G mutant by trypsin (protease) at 37° C. for an hour had not lead to protein fragmentation. In the case of recombinant wild-type FGF1 protein, the mutant was almost totally degraded by the protease. The obtained results prove a high proteolytic stability of the protein with substitution of three amino acids (Malgorzata Zakrzewska et al., 2005).

TABLE 3
Shortened list of denaturation temperatures of individual
FGF-1 protein muteins, determined using circular
dichroism (Malgorzata Zakrzewska et al., 2005)

| | Denaturation |
|---|---|
| | temperature |
| Mutant | T_{den} [° C.] |
| FGF-1 (wild) | 40.3 |
| Q55P | 48.1 |
| S62I | 49.3 |
| H108G | 48.0 |
| Q55P/S62I | 55.5 |
| Q55P/S62I/H108G | 61.8 |

In summary, current literature identifies a number of various directions in modifying the primary structure of FGF-1 protein in order to decrease its mitogenic potential, maintain or increase its biological activity to maintain homeostasis of cellular metabolism or to improve its physical and chemical, and consequently pharmacological properties. However, the research carried out so far has not made it possible to develop a mutein exhibiting the pharmacological properties and high efficacy expected from a therapeutic molecule. Therefore, there is still an urgent need to develop FGF-1 muteins which would be optimal from the point of view of pharmacotherapy (i.e. which would be characterized by a high thermal stability, resistance to proteolytic degradation and, as a result, longer half-life in an organism), which do not exhibit mitogenic potential and which, on one hand, show a high therapeutic effect, especially in the case of T2D (measured by effective reduction and stabilization of blood glucose level), and the same time do not pose a risk of hypoglycaemia.

### OBJECT OF THE INVENTION

The object of the invention is to provide human FGF-1 muteins and their constructs, such as dimers, which do not have the disadvantages known from the prior art. More specifically, the object of the invention is to provide human FGF-1 muteins which do not exhibit mitogenic potential. Furthermore, the object of the invention is to provide human FGF-1 muteins characterized by a high thermal stability, resistance to proteolytic degradation and consequently a longer half-life after administration to an organism. The object of the invention is also to provide human FGF-1 muteins having blood glucose level reducing action without a risk of causing hypoglycaemia, which are characterized by physical, chemical and pharmacological properties making them suitable for use in therapy, in particular in the treatment of type 2 diabetes.

These objects have been provided by the inventions defined in the attached patent claims.

### BRIEF DESCRIPTION OF THE INVENTION

According to the present invention and disclosure, there have been developed muteins of human FGF-1 protein and their dimers which comprise point mutations decreasing the mitogenic potential of human FGF-1 proteins. According to the invention and disclosure, there have also been developed muteins of human FGF-1 protein and their dimers preferably comprising additional point mutations which increase the stability of human FGF-1 muteins by increasing their denaturation temperature, as well as mutations due to which a longer half-life is obtained in the bloodstream after administration to a subject, due to increased resistance to proteolysis. Such properties enable efficient production process on a large scale and thus pharmacological development as a potential therapeutic, and prevent the protein from quick degeneration in an organism, which ensures a high therapeutic effect. Developed in accordance with the invention, human FGF-1 muteins comprising combinations of point mutations are unique, so far unknown in the literature, and provide non-obvious technical effects enabling effective therapeutic action thereof, especially for use in the treatment of diabetes, including type 2 diabetes.

The invention provides a human FGF-1 mutein having decreased mitogenicity, characterized in that it comprises two point mutations: at amino acid position S114 and at amino acid position L150, the numbering of amino acid positions being based on a full-length sequence of a wild-type FGF-1 protein as presented in Sequence No. 1 (SEK1).

Preferably, in the FGF-1 mutein according to the invention the point mutation at position S114 is S114A mutation (the mutein having Sequence No. 3 (SEK3).

Preferably, in the FGF-1 mutein according to the invention the point mutation at position L150 is L150D mutation (the mutein having Sequence No. 4 (SEK4).

More preferably, the FGF-1 mutein according to the invention comprises both S114A point mutation and L150D point mutation (the mutein having Sequence No. 5 (SEK5).

Preferably, the FGF-1 mutein according to the invention further comprises at least one stabilizing mutation at amino acid position selected from: Q55, S62 and H108.

More preferably, at least one stabilizing mutation at amino acid position Q55, S62 or H108 is a point mutation selected from: Q55P, S62I and H108G, respectively.

Preferably, the FGF-1 mutein according to the invention comprises three stabilizing mutations: Q55P, S62I and H108G.

More preferably, the FGF-1 mutein according to the invention has an amino acid sequence presented in Sequence No. 10 (SEK10).

Preferably, the FGF-1 mutein according to the invention additionally comprises an N-terminal deletion of at least 19 contiguous amino acids of the full-length FGF-1 protein.

More preferably, the FGF-1 mutein according to the invention comprises the N-terminal deletion of E3 to G21 amino acids of the full-length FGF-1 protein.

Preferably, the FGF-1 mutein according to the invention comprises S114A point mutation, L150D point mutation and the N-terminal deletion of E3 to G21 amino acids of the full-length FGF-1 protein.

More preferably, the FGF-1 protein according to the invention has an amino acid sequence presented in Sequence No. 18 (SEK18).

The invention further provides a dimer of the mutated FGF-1 protein having decreased mitogenicity according to the invention as defined above.

Preferably, the dimer according to the invention is a homodimer.

More preferably, muteins forming the dimer according to the invention are connected by a linker, more preferably by an amino acid linker, most preferably such a linker is an amino acid sequence GGGGSGGGGSGGGG.

Even more preferably, the dimer according to the invention has an amino acid sequence presented in Sequence No. 21 or 22 (SEK21 or SEK22).

The invention further provides the human FGF-1 mutein having decreased mitogenicity according to the invention as defined above for use in reducing blood glucose level.

The invention further provides the human FGF-1 mutein having decreased mitogenicity according to the invention as defined above for use in the treatment of diabetes, in particular type 2 diabetes.

Preferably, the FGF-1 mutein for use according to the invention has an amino acid sequence presented in Sequence No. 10 (SEK10).

Preferably, the FGF-1 mutein for use according to the invention has an amino acid sequence presented in Sequence No. 18 (SEK18).

The invention also provides the dimer of human FGF-1 muteins having decreased mitogenicity according to the invention as defined above for use in reducing blood glucose level.

The invention also provides the dimer of human FGF-1 muteins having decreased mitogenicity according to the invention as defined above for use in the treatment of diabetes, in particular type 2 diabetes.

Preferably, the dimer for use according to the invention has an amino acid sequence presented in Sequence No. 21 (SEK21).

Preferably, the dimer for use according to the invention has an amino acid sequence presented in Sequence No. 22 (SEK22).

Disclosed herein is also a mutein of human fibroblast growth factor 1 (FGF-1) having decreased mitogenicity, characterized in that it comprises a point mutation at amino acid position S153, the numbering of amino acid positions being based on a sequence of a full-length wild-type FGF-1 protein as presented in Sequence no 1.

Such a point mutation at position S153 is S153A mutation (the mutein having Sequence No. 32 (SEK32) or S153R mutation (the mutein having Sequence no 31 (SEK31).

Such a human FGF-1 mutein according to the disclosure may additionally comprise a S154 point mutation. In such FGF-1 mutein according to the disclosure the point mutation at position S154 is S154D mutation, and the point mutation at position S153 is S153D point mutation (such a mutein is presented in Sequence No. 27).

Such a human FGF-1 mutein according to the invention optionally further comprises at least one stabilizing mutation at amino acid position selected from: Q55, S62 and H108. Such at least one stabilizing mutation at amino acid position Q55, S62 or H108 is a point mutation selected from: Q55P, S62I and H108G, respectively; such FGF-1 mutein according to the disclosure comprises in particular three stabilizing mutations: Q55P, S62I and H108G; and especially such FGF-1 mutein according to the invention has an amino acid sequence presented in Sequence No. 23, Sequence No. 25 or Sequence No. 29.

Optionally such FGF-1 mutein according to the disclosure additionally comprises an N-terminal deletion of at least 19 contiguous amino acids of the full-length FGF-1 protein, in particular the N-terminal deletion of E3 to G21 amino acids of the full-length FGF-1 protein. Such FGF-1 mutein according to the disclosure alternatively comprises: S153A point mutation, S153R point mutation or a combination of S153D and S154D point mutations, and the N-terminal deletion of E3 to G21 amino acids of the full-length FGF-1 protein, and in particular such a mutein according to the disclosure has an amino acid sequence presented in Sequence No. 24, Sequence No. 26, Sequence No. 30, Sequence No. 33 or Sequence No. 34.

Disclosed herein is also a dimer of human fibroblast growth factor (FGF-1) muteins having decreased mitogenicity and comprising a mutation at position S153 as defined above, including a homodimer.

Muteins forming such a dimer are connected by a linker, particularly an amino acid linker, in particular a linker which is an amino acid sequence GGGGSGGGGSGGGG.

Such a dimer according to the disclosure has an amino acid sequence selected from sequences with numbers 35 to 38 (SEK35 to SEK38).

Disclosed herein is also a human fibroblast growth factor 1 (FGF-1) mutein having decreased mitogenicity comprising a mutation at position S153, and optionally at position S154, as defined above for use in reducing blood glucose level.

Disclosed herein is also a human fibroblast growth factor 1 (FGF-1) mutein having decreased mitogenicity comprising a mutation at position S153, and optionally at position S154 as defined above for use in the treatment of diabetes, in particular type 2 diabetes.

Such human FGF-1 mutein has a sequence of amino acids selected from Sequences Nos. 23 to 30.

### DETAILED DESCRIPTION OF THE INVENTION

In the first aspect the present invention provides a mutated protein (mutein) of human fibroblast growth factor 1 (FGF-1) with decreased, relative to wild-type FGF-1, mitogenicity. Such a mutated protein of human FGF-1 is interchangeably referred to herein as human FGF-1 mutein or human FGF-1 mutant. Methods of introducing point mutations into protein sequences and tests for mitogenicity of protein agents are known in the art and preferable examples of such methods and tests suitable to carry out the present invention are presented below. Human FGF-1 mutein according to the first aspect comprises at least two point mutations, namely a mutation of naturally occurring amino acid S at position 114 of the full-length protein (155 aa) and a mutation of naturally occurring amino acid L at position 150 of the full-length FGF-1 protein as presented in Sequence 1.

Point mutations at positions S114 and L150 of the full-length wild-type human FGF-1 protein cause decreased mitogenicity of the FGF-1 mutein.

Preferably, the FGF-1 mutein according to the invention comprises S114A mutation:

The above point mutation transcript is a standard method of designating point mutations in proteins and in this case it denotes a point mutation which consists in replacing the amino acid—serine—naturally occurring in the polypeptide chain of the human wild-type FGF-1 protein at position 114, with another amino acid—alanine (A).

S114A mutation reduces affinity of the mutated protein to CK2 kinase and leads to decreased mitogenicity of cells compared to the full-length wild-type human FGF-1 protein, as indicated in the embodiments below and presented in the Figures (e.g. FIG. 2).

Preferably, the variant of the full-length FGF-1 mutein according to the first aspect of the present invention comprises L150D mutation. L150D mutation reduces binding of the mutated FGF-1 protein according to the invention to the FGFR1 receptor. This mutation is located in a domain responsible for affinity to the receptor, but it is not crucial for this binding. FIG. 3. shows the reduced binding to the FGFR1 receptor of the human FGF-1 mutein comprising L150D mutation. Such protein is characterized by decreased mitogenicy, as demonstrated below and presented in the Figures (e.g. FIG. 3).

More preferably, such FGF-1 mutein according to the invention comprises both S114A mutation and L150D mutation. Such FGF-1 proteins according to the invention are characterized by the optimal reduction of mitogenicity, as indicated below and presented in the Figures (e.g. FIG. 4).

Guided by the insight into the structure of the FGF1 protein, the inventors have designed and have obtained an FGF1 mutein with reduced ability to bind to FGFR 1-4 receptors. The inventors have achieved this by replacement Leu150Asp (L150D), a well-known amino acid residue responsible for direct physical interaction with FGFR. A reduction of binding affinity to the FGFR leads to reduction of mitogenic potential of the protein, but also reduction of its thermal stability. A similar effect on protein stability had Ser114Ala (S114A) replacement. This point mutation is responsible for reduction of the physical interaction with proteins CK2 and p34 and is a homolog of the well-known non-mitogenic FGF2^{(S117A)} mutant protein. CK2 and p34 are required, respectively, for phosphorylation and nuclear transport of exogenous FGF1, and consequently the position of S114 is a factor responsible for activation of mitogenicity. Lack of phosphorylation of FGF1 at the S114A position decreases mitogenic properties, and thus the introduction of S114A substitution increases the therapeutic potential of FGF1. Unfortunately, both substitutions-S114A and L150D-reduced thermal stability, compared to the FGF1^{(WT)}, which makes it difficult to produce them on a large scale. Based on the above findings, the inventors combined mutations S114A/L150D with the very well characterized, stable triple mutation: Gln55Pro/Ser62Ile/His108Gly (Q55P/S62I/H108G), also known as 3×. Mutations at these residues radially increase stability of the protein, but also its mitogenic potential. Having introduced the fivefold 3×/S114A/L150D substitution, the inventors obtained new FGF1^{(3x/S114A/L150D)}. Due to the specific combination of the 5-fold substitution the inventors obtained a thermally stable protein exhibiting a significantly increased glucose uptake, both in vitro and in vivo, and not exhibiting mitogenicity.

Thus, preferably, the present invention provides the human FGF-1 mutein having decreased mitogenicity, as described above, which additionally comprises at least one stabilizing point mutation at amino-acid position: Q55, S62 and H108 (according to the numeration of the amino acid positions of the full-length human wild-type protein—the sequence identification no. 1); more preferably, the mutations are selected from: Q55P, S62I and H108G. Even more preferably, the FGF-1 mutein according to the invention comprises the additional mutations at all three positions. Methods for determining stability of FGF-1 proteins are known in the art and an exemplary method for determining their stability is presented below and in Table 3.

Such FGF-1 muteins according to the invention are characterized by both decreased mitogenicity and also increased stability, resistance to proteolysis, longer half-life after administration, and they effectively reduce blood glucose level, without the simultaneous induction of hypoglycaemia; thus they exhibit an optimal antidiabetic action.

More preferably, such FGF-1 muteins according to the first aspect of the present invention further comprise a deletion of at least 19 amino acids from the N-terminus of the protein, in particular a deletion of E3-G21 amino acids (from N-terminus), which additionally reduces mitogenicity.

The amino acid sequence of the polypeptide chain of the FGF1 protein truncated at N-terminus, in a version without mutations, on the template of which there were performed the amino acid substitutions according to the invention, in the truncated version (proteins/variants described as “short”—the truncated (shortened) sequence, i.e. with a deletion of at least 19 amino acids from N-terminus), has the total chain length of 136 aa. Sequence No. 2 (SEK2) as compared to the full-length wild-type FGF-1 protein, lacks E3-G21 amino acids (i.e. amino acids EGEITTFTALTEKFNLPPG). According to the invention the truncated version of the human FGF-1 protein is also called the short variant of the human FGF-1 protein, preferably consisting of 136 aa, whereas the full-length version of the human FGF-1 protein is also called the long variant of the human FGF-1 protein, which consists of 155 aa and does not comprise the deletion of E3-G21 amino acids.

Considering the pharmacologically advantageous parameters of such a mutein of human FGF-1, apart from the decreased mitogenicity, (i.e. achieved higher thermal stability and longer half-life with the presence of heparin), a triple stabilizing mutation was also tested in an in vivo test. As it turned out, such human FGF-1 muteins comprising the indicated 5 point mutations according to the invention also exhibit antidiabetic action. Due to the above described properties of FGF1 muteins according to this aspect of the invention, they may be effectively used in medicine, in particular for reducing blood glucose levels, especially in the treatment of diabetes, including type 2 diabetes.

In the second aspect, the present invention provides a dimer of any of the above described human FGF-1 muteins having decreased mitogenicity according to the invention. Methods of producing dimers of proteins are known in the art, and a preferable method to obtain dimers of muteins according to the present inventions is described below. Preferably, the dimer according to the invention is a homodimer of any of the above described human FGF-1 muteins having decreased mitogenicity according to the invention, more preferably connected by a linker, in particular an amino-acid linker. This makes it possible to obtain physical and chemical stabilization facilitating dimerization of a ligand. A ligand dimer more easily induces dimerization of cell receptors. Additionally, this allows one to increase the protein mass, because proteins with masses over 30-40 kDa are not quickly eliminated from an organism through kidneys and have a longer half-live. Even more preferably, such a linker is the amino acid sequence GGGGSGGGGGGGG. Particularly advantageous variants of dimers of human FGF-1 muteins according to the invention have amino acid sequences as presented in Sequence no 21 and 22 (SEK21 and SEK22)).

The properties of the above described human FGF-1 muteins according to the invention enable their use in medicine, in particular for reducing blood glucose level, and particularly in the treatment of diabetes, as is demonstrated below.

Human FGF-1 muteins and dimers thereof according to the invention do not have the undesirable features known from the prior art and that is why they can be used in medicine, pharmacy, and in a therapy, as medicaments. They are characterized by decreased mitogenicity and preferably increased thermal stability and increased resistance to proteolysis, as well as suitable pharmacological properties. Additionally, they are preferably characterized by a blood glucose level reducing action without posing a risk of hypoglycaemia, and thus they can be used in particular for reducing glucose level, especially in the treatment of diabetes, in particular type 2 diabetes.

Disclosed herein is also a mutein of human fibroblast growth factor 1 (FGF-1) having decreased mitogenicity which comprises a point mutation at amino acid position S153, the numbering of amino acid positions being based of the full-length wild-type FGF-1 protein sequence, as presented in Sequence No. 1. Such human FGF-1 mutein according to the herein described aspect of the disclosure comprises a point mutation selected from: S153A mutation (the mutein having Sequence No. 32 (SEK32) and S153R mutation (the mutein having Sequence No. 31 (SEK31).

More preferably, such human FGF-1 mutein according to the disclosure additionally comprises a point mutation at amino acid position S154. Even more preferably, such a mutation is provided by S154D mutation, wherein the point mutation at position S153 is provided by mutation S153D (such a mutein is shown in Sequence No. 27 (SEK27). The above mentioned mutations at positions S153 and S154 are located in a domain responsible for affinity of the ligand to the receptor. Human FGF-1 muteins according to the herein described aspect of the disclosure are characterized by decreased mitogenicity and effective reduction of blood glucose level and thus they can be used for reducing blood glucose level, particularly in the treatment of diabetes, including type 2 diabetes.

Preferably, such human FGF-1 mutein according to the disclosure further comprises at least one stabilizing mutation at an amino-acid position selected from: Q55P, S62I and H108G, particularly three such stabilizing mutations (Q55P, S62I and H108G). Most preferably, such FGF-1 mutein according to the herein described aspect of the present disclosure has an amino-acid sequence presented is Sequence No. 23 (SEK23), Sequence No. 25 (SEK25) or Sequence No. 29 (SEK29). Such variants are characterized by the optimal reduction of mitogenicity, optimal thermal stability and they exhibit a glucose level reducing potential, and consequently they exhibit antidiabetic action. Even more preferably, such muteins according to the disclosure may also comprise N-terminal deletion of at least 19 amino acids of the full-length human protein (the short variant), particularly E3 to G21 amino acids of the full-length human FGF-1 protein. Methods for determining stability of FGF-1 proteins are known in the art, and an exemplary method for determining their stability is presented below.

Disclosed herein is also a dimer of human fibroblast growth factor (FGF-1) muteins having decreased mitogenicity comprising a mutation at position S153, and optionally S154, as described above. Dimers of human FGF-1 muteins according to this aspect of the disclosure are obtained in an analogous way as dimers of the muteins according to the first aspect of the present invention. Preferably, such a dimer is a homodimer of any of the above described human FGF-1 muteins having decreased mitogenicity according to the herein described aspect of the disclosure, more preferably connected by a linker, in particular an amino acid linker, which most preferably is the amino-acid sequence GGGGSGGGGSGGGG. This enables obtaining the physical and chemical stabilisation facilitating ligand dimerization. Such a dimer more easily induces dimerization of cellular receptors. Additionally, the formation of a dimeric form makes it possible to increase the protein mass, which is desirable because proteins with masses over 30-40 kDa are not quickly eliminated from an organism through kidneys and have a longer half-live. Particularly advantageous variants of dimers of human FGF-1 muteins according to this aspect of the disclosure have amino acid sequences selected from the sequences presented in Sequences Nos. 35 to 38.

The muteins according to the herein described aspect of the present disclosure have properties which make them suitable for use in reducing blood glucose level, in particular in the treatment of diabetes, particularly type 2 diabetes.

The invention will now be illustrated in the following figures and following examples, which however are not intended to limit, in any way, the scope of the invention as defined in the patent claims. Unless indicated otherwise, all methods, reactants and parameters are the same as those commonly used in the field to which the present invention belongs and which are recommended by the manufacturers thereof.

### Description of the List of Sequences

All amino acid sequences included in the patent claims and in this description are based on the cDNA FGF1_WT sequence, Wild-type variant, GenBank accession number NM 001354952.2, encoding human fibroblast growth factor 1 (FGF-1) having a total length of 155 amino acids.

### Sequence No. 1 (SEK1)

Based on the above sequence, a full version of FGF1 (1-155aa), named herein as FGF1(155aa), was produced.

```
(SEK1)
FGF1 (1-155 aa)
FGF1^{(155 aa)}
FGF_WT
Sequence No. 1
                10 20 30 40

[symbol]

[symbol]

[symbol]

[symbol]

                50 60 70 80

[symbol]

[symbol]

[symbol]

[symbol]

                90 100 110 120

[symbol]

[symbol]

[symbol]

[symbol]

              130 140 150

[symbol]

[symbol]

[symbol]

[symbol]

[symbol]

indicates data missing or illegible when filed
```

### Sequence No. 2 (SEK2)

Based on the above sequence, a truncated version of FGF1 (22-155aa), named herein as FGF1(Δ155aa), was produced. The protein was generated by deletion of a fragment of N′ terminus. In the sequence, the bold font indicates first 2 amino acids whose presence results from the method of cloning of the truncated FGF1 (aa 22-155) sequence into an expression vector. Methionine (M) is encoded by the start codon AUG, from which the protein expression process starts, and the presence of which is required at N′ terminus. Alanine (A) was added to the sequence as a result of adding two nucleotides to the sequence flanking the restriction site in order to preserve an adequate reading frame in the transcription process. The addition of Alanine does not cause any physical and chemical changes in the protein due to its nonpolar and aliphatic nature.

(SEK2)
FGF1 (22-155 aa)
FGF1^{(Δ155 aa)}
FGF_ΔWT
Sequence No. 2
MA-------- ---------- -

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

indicates data missing or illegible when filed

### Sequence No. 3 (SEK3)

Mutant full-length FGF1 (1-155aa) sequence, wherein Alanine (A) was substituted for Serine(S) at position 114. FGF1(155aa; S114A) point mutation. Serial No. of the mutation: M1.

| | |
|---|---|
| Sequence No. 3 (SEK3) | MAEGEITTFT ALTEKFNLPP GNYKKPKLLY CSNGGHFLRI LPDGTVDGTR DRSDQHIQLQ |
| FGF1 (1-155aa) | 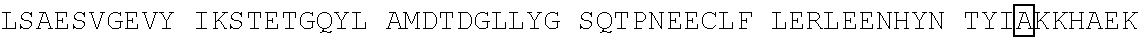 |
| FGF1^{(155aa;S114A)} | NWFVGKKNG SCKRGPRTHY GQKAILFLPL PVSSD |
| M1 | |

### Sequence No. 4 (SEK4)

Mutant full-length FGF1 (1-155aa) sequence, wherein at position 150, Aspartic acid (D) was substituted for Leucine (L). FGF1(155aa; L150D) point mutation. Serial No. of the mutation: M2.

| | |
|---|---|
| Sequence No. 4 (SEK4) | MAEGEITTPT ALTEKFNLPP GNYKEPKLLY CSWGGNFLRI LPDGTVGGTA DP [symbol] DQHIQLQ |
| FGF1 (1-155aa) | LSAESVGEVY IKDTEICQYL AHEYDGGLLYG SQTPSEECLF LERLEEN [symbol] YN TYISKKHAEK |
| FGF1 [symbol] | 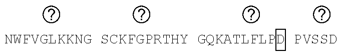 |
| M2 | |

[symbol]

indicates data missing or illegible when filed

### Sequence No. 5 (SEK5)

Mutant full-length FGF1 (1-155aa) sequence, wherein at position 114, Alanine (A) was substituted for Serine(S)—S114A, and at position 150, Aspartic acid (D) was substituted for Leucine (L)—L150D. FGF1(155aa; S114A/L150D) double mutation. Serial No. of the mutation: M3.

| | |
|---|---|
| | 10 20 30 40 50 60 |
| Sequence No. 5 (SEK5) | MAEGEITTFT ALTEKFNLPP NYKKPKLLY CSNGGHFLRI LPDGTVDGTR DRSDQHIQLQ |
| | 70 80 90 100 110 120 |
| FGF1 (1-155aa) | 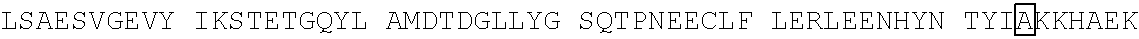 |
| | 130 140 150 |
| FGF1^{(155aa;S114A/L150D)} | 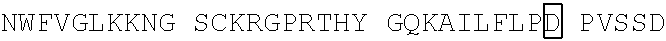 |
| M3 | |

### Sequence No. 6 (SEK6)

Mutant full-length FGF1 (1-155aa) sequence, wherein at position 55, Proline (P) was substituted for Glutamine (Q). FGF1(155aa; Q55P) point mutation. Serial No. of the mutation: M4.

| | |
|---|---|
| Sequence No. 6 (SEK6) | 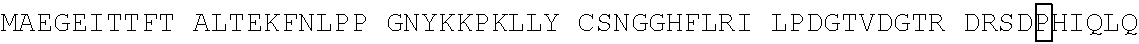 |
| FGF1 (1-155aa) | LSAESVGEVY IKSTETGQYL AMDTDGLLYG SQTPNEECLF LERLEENHYN TYISKKHAEK |
| FGF1^{(155aa;Q55P)} | NWFVGLKKNG SCKRGPRTHY GQKAILFLPL PVSSD |
| M4 | |

### Sequence No. 7 (SEK7)

Mutant full-length FGF1 (1-155aa) sequence, wherein at position 62, Isoleucine (I) was substituted for Serine(S). FGF1(155aa; S62I) point mutation. Serial No. of the mutation: M5.

| | |
|---|---|
| Sequence No. 7 (SEK7) | MAEGEITTFT ALTEKFNLPP GNYKKPKLLY CSNGGHFLRI LPDGTVDGTR DRSDQHTQLQ |
| FGF1 (1-155aa) | 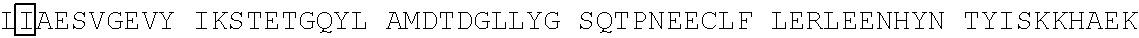 |
| FGF1^{(155aa;S62I)} | NWFVGLKKNG SCNRGPRTHY GQKAILFLPL PVSSD |
| M5 | |

### Sequence No. 8 (SEK8)

Mutant full-length FGF1 (1-155aa) sequence, wherein at position 108, Glycine (G) was substituted for Histidine (H). FGF1(155aa; L108G) point mutation. Serial No. of the mutation: M6.

| | |
|---|---|
| Sequence No. 8 (SEK8) | MAEGEITTFT ALTEKFNLPP GNYKKPKLLY CSNGGHFLRI LPDGTVDGTR DRSDQHIQLQ |
| FGF1 (1-155aa) | 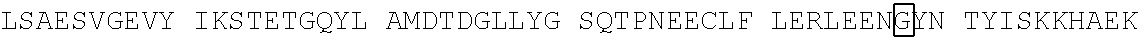 |
| FGF1^{(155aa;H108G)} | NWFVGLKKNG SCKRGPRTHY GQKAILFLPL PVSSD |
| M6 | |

### Sequence No. 9 (SEK9)

Mutant full-length FGF1 (1-155aa) sequence, wherein at position 55, Proline (P) was substituted for Glutamine (Q)—Q55P, and at position 62, Isoleucine (I) was substituted for Serine(S)—S62I, and at position 108, Glycine (G) was substituted for Histidine (H)—H108G. FGF1(155aa; Q55P/S62I/H108G) triple mutation. Serial No. of the mutation: M7.

| | |
|---|---|
| | 10 20 30 40 50 60 |
| Sequence No. 9 (SEK9) | 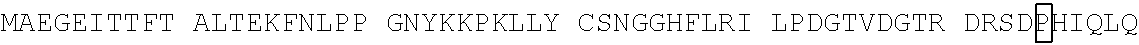 |
| | 70 80 90 100 110 120 |
| FGF1 (1-155aa) | 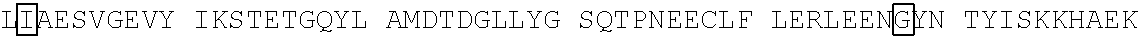 |
| | 130 140 150 |
| FGF1^{(155aa;Q55P/S62I/H108G)} | NWFVGLKKNG SCKRGPRTHY GQKAILFLPL PVSSD |
| M7 | |

### Sequence No. 10 (SEK10)

Mutant full-length FGF1 (1-155aa) sequence, wherein at position 55, Proline (P) was substituted for Glutamine (Q)—Q55P, and at position 62, Isoleucine (I) was substituted for Serine(S)—S62I, and at position 108, Glycine (G) was substituted for Histidine (H)—H108G; additionally, at position 114, Alanine (A) was substituted for Serine(S)—S114A, and at position 150, Aspartic acid (D) was substituted for Leucine (L) L150D. FGF1(155aa; Q55P/S62I/H108G/S114A/L150D) fivefold mutation. Serial No. of the mutation: M8.

| | |
|---|---|
| Sequence No. 10 (SEK10) | 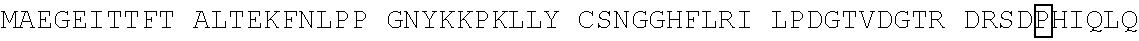 |
| FGF1 (1-155aa) | 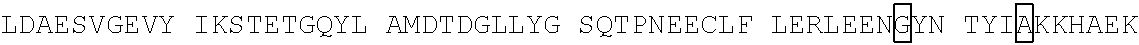 |
| FGF1^{(155aa;Q55P/S621I/H108G/S114A/L150D)} | 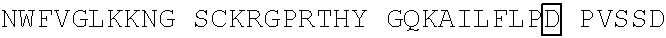 |
| M8 | |

### Sequence No. 11 (SEK11)

Mutant truncated FGF1 (22-155aa) sequence, wherein at position 114, Alanine (A) was substituted for Serine(S). FGF1(Δ155aa; S114A) point mutation. Serial No. of the mutation: M9.

| | |
|---|---|
| Sequence No. 11 (SEK11) | MA-------- ---------- -NYKPKKLLY CSNGGHFLRI LPDGTVDGTR DRSDQHIQLQ |
| FGF1 (22-155aa) | 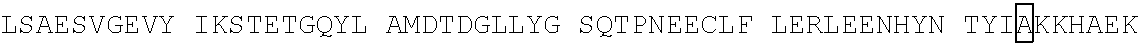 |
| FGF1^{(Δ155aa;S114A)} | NWFVGLKKNG SCKRGPRTHY GQKAILFLPL PVSSD |
| M9 | |

### Sequence No. 12 (SEK12)

Mutant truncated FGF1 (22-155aa) sequence, wherein at position 150, Aspartic acid (D) was substituted for Leucine (L). FGF1(Δ155aa; L150D) point mutation. Serial No. of the mutation: M10.

| | |
|---|---|
| Sequence No. 12 (SEK12) | MA-------- ---------- -NYKKPKLLY CSNGGKFLRI LFDGTVDGTR DRSDQHIQLQ |
| FGF1 (22-155a) | LSAESVGEVY IKSTETGQYL AMDTDFLLYG SQTPNEECLF LERLEENHYN TYISKKHAEK |
| FGF1^{(Δ155aa;L150D)} | 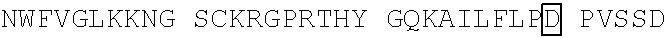 |
| H10 | |

### Sequence No. 13 (SEK13)

Mutant truncated FGF1 (22-155aa) sequence, wherein at position 114, Alanine (A) was substituted for Serine(S) S114A and at position 150, Aspartic acid (D) was substituted for Leucine (L) L150D. FGF1(Δ155aa; S114A/L150D) d mutation. Serial No. of the mutation: M11.

| | |
|---|---|
| | 10 20 30 40 50 60 |
| Sequence No. 13 (SEK13) | MA-------- ---------- -NYKKPKLLY CSNGGHFLRI LPDGTVDGTR DRSDQHIQLQ |
| | 70 80 90 100 110 120 |
| FGF1 (22-155aa) | 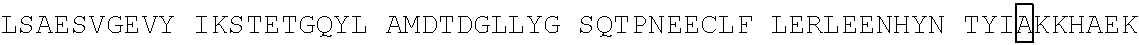 |
| | 130 140 150 |
| FGF1^{(Δ155aa;S114A/L150D)d} | 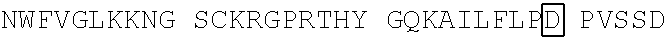 |
| M11 | |

### Sequence No. 14 (SEK14)

Mutant truncated FGF1 (22-155aa) sequence, wherein, at position 55, Proline (P) was substituted for Glutamine (Q). Point mutation FGF1(Δ155aa; Q55P). Serial No. of the mutation: M12.

| | |
|---|---|
| Sequence No. 14 (SEK14) | 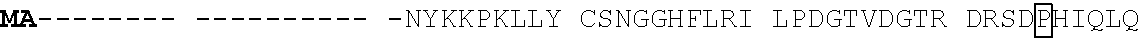 |
| FGF1 (22-155aa) | LSAESVGEVY IKSTETGQYL AMDTDGLLYG SQTPNEECLF LERLEENHYN TYISKKHAEF |
| FGF1^{(Δ155aa;Q55P)} | NWFVGLKKNG SCKRGPRTHY GQKAILFLPL PVSSD |
| M12 | |

### Sequence No. 15 (SEK15)

Mutant truncated FGF1 (22-155aa) sequence, wherein, at position 62, Isoleucine (I) was substituted for Serine(S). FGF1(Δ155aa; S62I) point mutation. Serial No. of the mutation: M13.

| | |
|---|---|
| Sequence No. 15 (SEK15) | MA-------- ---------- -NYKKPKLLY CSNGGHFLRI LPDGTVDGTR DRSDQHIQLQ |
| FGF1 (22-155a) | 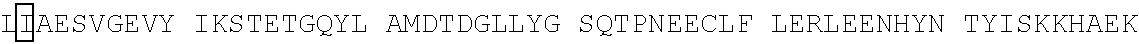 |
| FGF1^{(Δ155aa;S62I)} | NWFVGLKKNG SCKRGPRTHY GQKAILFLPL PVSSD |
| M13 | |

### Sequence No. 16 (SEK16)

Mutant truncated FGF1 (22-155aa) sequence, wherein, at position 108, Glycine (G) was substituted for Histidine (H). FGF1(Δ155aa; H108G) point mutation. Serial No. of the mutation: M14.

| | |
|---|---|
| Sequence No: 16 (SEK16) | MA-------- ---------- -NYKKPKLLY CSNGGHFLRI LPDGTVDGTR DRSDQHIQLQ |
| FGF1 (22-155aa) | 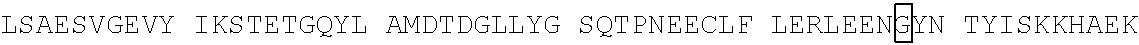 |
| FGF1^{(Δ155aa;H108G)} | NWFVGLKKNG SCKRGPRTHY GQKAILFLPL PVSSD |
| M14 | |

### Sequence No. 17 (SEK17)

Mutant truncated FGF1 (22-155aa) sequence, wherein, at position 55, Proline (P) was substituted for Glutamine (Q)—Q55P and at position 62, Isoleucine (I) was substituted for Serine(S)—S62I and at position 108, Glycine (G) was substituted for Histidine (H)—H108G. FGF1(Δ155aa; Q55P/S62I/H108G) triple mutation. Serial No. of the mutation: M15.

| | |
|---|---|
| | 10 20 30 40 50 60 |
| Sequence No. 17 (SEK17) | 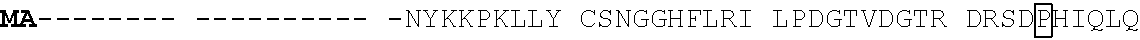 |
| | 70 80 90 100 110 120 |
| FGF1 (22-155aa) | 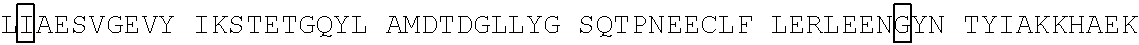 |
| | 130 140 150 |
| FGF1^{(Δ155aa;Q55P/S62I/H108G)} | NWFVGLKKNG SCKRGPRTHY GQKAILFLPL PVSSD |
| M15 | |

### Sequence No. 18 (SEK18)

Mutant truncated FGF1 (22-155aa) sequence, wherein, at position 55, Proline (P) was substituted for Glutamine (Q) Q55P and at position 62, Isoleucine (I) was substituted for Serine(S)—S62I, and at position 108, Glycine (G) was substituted for Histidine (H)—H108G; additionally, at position 114, Alanine (A) was substituted for Serine(S)—S114A, and at position 150, Aspartic acid (D) was substituted for Leucine (L)—L150D. FGF1(Δ155aa; Q55P/S62I/H108G/S114A/L150D) fivefold mutation. Serial No. of the mutation: M16.

| | |
|---|---|
| Sequence No. 28 (SEK18) | 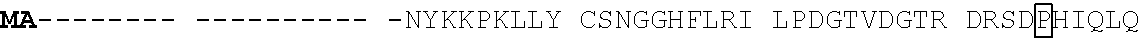 |
| FGF1 (22-155aa) | 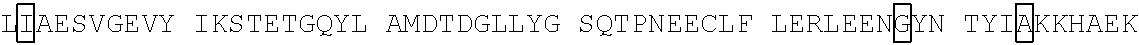 |
| FGF1^{(Δ155aa;Q55P/S62I/H108G/S114A/L150D)} | 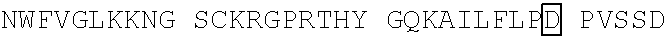 |
| M16 | |

### Sequence No. 19 (SEK19)

Homodimer of the full-length FGF1 (1-155 aa), wherein two Sequences No. 1 were connected by the linker: GGGGSGGGGSGGGG N′-terminus-(1-155-linker-2-155)-C′-terminus. The second FGF1(155aa) monomer starts with Alanine (A) at position 170, Methionine (M) was omitted. FGF1_DIMER(155aa).

| | |
|---|---|
| | 10 20 30 40 50 60 |
| Sequence No. 19 (SEK19) | MAEGETTTFT ALTEKFNLPP GNYKKPKLLY CSNGGHFLRI LPDGTVDGTR DRSDQHIQLQ |
| | 70 80 90 100 110 120 |
| FGF1 (1-155aa) | LSAESVGEVY IKSTETGQYL AMDTDGLLYG SQIPNEECLF LERLEENHYN TYISKKHAEK |
| | 130 140 150 160 170 180 |
| FGF1_DIMER^{(155aa)} | 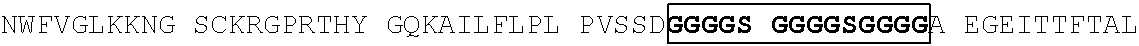 |
| | 190 200 210 220 230 240 |
| FGF1_WT_DIMER | TEKFNLPPNV KKPKLLYGSN GGHFLRILPD GTVDGTRDRS DQNIQLQLGA ESVGEVYIKS |
| | 250 260 270 280 290 300 |
| | TETGQYLAMD YDGLLYGGQT PNEECLFLEP LEENHYNTYI SKKHAEKNWF VGLKKNGSCK |
| | 310 320 |
| | RGPPTHYGQK ATLFLPLPVS SD |

### Sequence No. 20 (SEK20)

Homodimer of the truncated FGF1 (22-155 aa), wherein two Sequences No. 2 were connected by the linker: GGGGSGGGGSGGGG N′-terminus-(22-155-linker-22-155)-C′-terminus. In the sequence, the bold font indicates the first 2 amino acids of the first FGF1(Δ155aa) monomer and the first amino acid of the second FGF1(Δ155aa) monomer, the presence of which results from the method of cloning of the truncated FGF1 (aa 22-155) sequence into an expression vector. Methionine (M) is encoded by the start codon AUG, from which the protein expression process starts and the presence of which is required at N′ terminus; Alanine (A) was added to the sequence as a result of adding two nucleotides to the sequence flanking the restriction site in order to preserve an appropriate reading frame in the transcription process. The addition of Alanine does not cause any physical and chemical changes in the protein due to its nonpolar and aliphatic nature. The second FGF1(Δ155aa) monomer starts with Alanine (A) at position 70; Methionine (M) was omitted. FGF1_DIMER(Δ155aa).

| | |
|---|---|
| Sequence No. 20 (SEK20) | MA-------- ---------- -NYKKPKLLY CSNGGHFLRI LPDGTVDGTR DRSDQHIQLQ |
| FGF1 (22-155aa) | LSAESVGEVY IKSTETGQYL AMDTDGLLYG SQIPNEECLF LERLEENHYN TYISKKHAEK |
| FGF1_DIMER^{(Δ155aa)} | 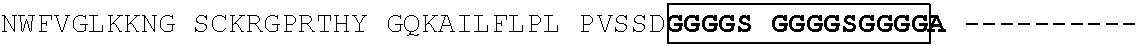 |
| FGF1_ΔWT_DIMER | --------NY KKPKLLYCSN GGHFLRILPD GTVDGTRDRS DQHIQLQLSA ESVGEVYIKS |
| | TETGQYLAMD TDGLLYSSQT PNEECLPLER LEENHYNTYI SKKHAEKNWF VGLKKNGSCK |
| | RGPRTHYGQK AILFLPLPVS SD |

### Sequence No. 21 (SEK21)

Homodimer of the full-length FGF1 (aa 1-155) mutation with the serial number M8, wherein two Sequences no 10 were connected by the linker: GGGGSGGGGSGGGG N′-terminus-(1-155-linker-2-155)-C′ terminus. The second FGF1(155aa; Q55P/S62I/H108G/S114A/L150D) monomer starts with Alanine (A) at position 170. Methionine (M) was omitted. FGF1_M8_DIMER(155aa).

| | |
|---|---|
| Sequence No. 21 (SEK21) | 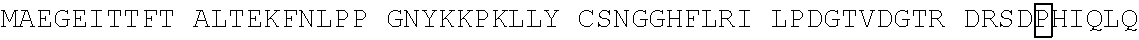 |
| FGF1 (1-155aa) | 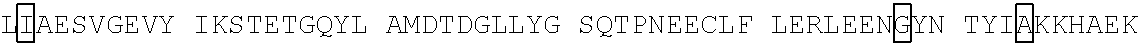 |
| FGF1_M8_DIMER^{(155aa)} | 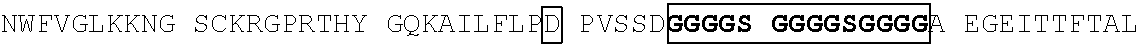 |
| | 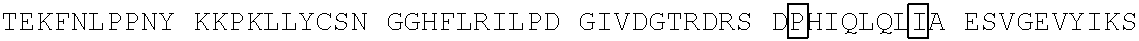 |
| | 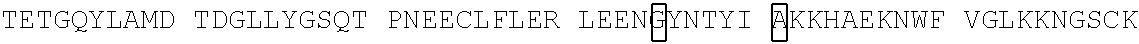 |
| | 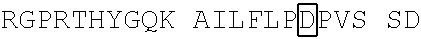 |

### Sequence No. 22 (SEK22)

Homodimer of the truncated FGF1 (aa 22-155), wherein two Sequences No. 18 were connected by the linker: GGGGSGGGGSGGGG N′ terminus-(22-155-linker-22-155)-C′ terminus. In the sequence, the bold font indicates the first two amino acids of the first FGF1(Δ155aa) monomer and the first amino acid of the second FGF1(Δ155aa; Q55P/S62I/H108G/S114A/L150D) monomer, the presence of which results from the method of cloning of the truncated FGF1(aa 22-155) sequence into an expression vector. Methionine (M) is encoded by the start codon AUG, from which the protein expression process starts, and the presence of which is required at N′ terminus; Alanine (A) was added to the sequence as a result of adding two nucleotides to the sequence flanking the restriction site in order to preserve an adequate reading frame in the transcription process. The addition of Alanine does not cause any physical and chemical changes of the protein due to its nonpolar and aliphatic nature. The second FGF1(Δ155aa; Q55P/S62I/H108G/S114A/L150D) monomer starts with Alanine (A) at position 170. Methionine (M) was omitted. FGF1_M16_DIMER(Δ155aa).

| | |
|---|---|
| Sequence No. 22 (SEK22) | 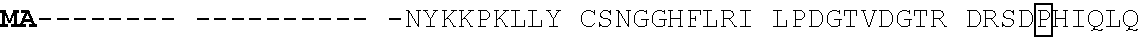 |
| FGF1 (22-155aa) | 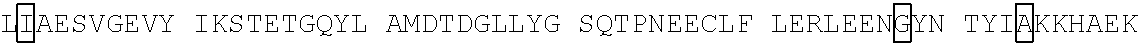 |
| FGF1_M16_DIMER^{(Δ155aa)} | 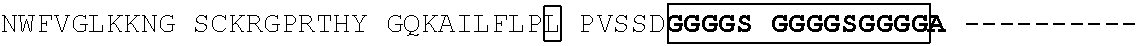 |
| | 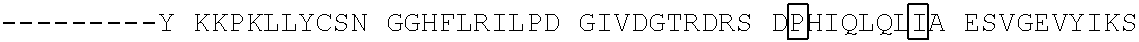 |
| | 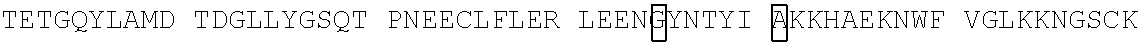 |
| | 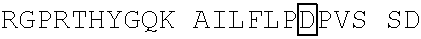 |

### Sequence No. 23 (SEK23)

Mutant full-length FGF1 (1-155aa) sequence, wherein, at position 55, Proline (P) was substituted for Glutamine (Q)—Q55P, and at position 62, Isoleucine (I) was substituted for Serine(S)—S62I, at position 108, Glycine (G) was substituted for Histidine (H)—H108G; additionally, at position 153, Serine(S) was substituted for Alanine (A)—S153A. FGF1(155aa; Q55P/S62I/H108G/S153A) fourfold mutation. Serial No. of the mutation: M17.

| | |
|---|---|
| Sequence No. 23 (SEK23) | 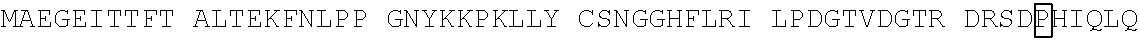 |
| FGF1 (1-155aa) | 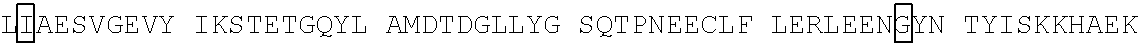 |
| FGF1_M^{(155aa)} | 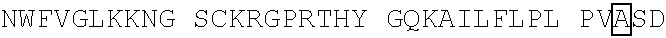 |

### Sequence No. 24 (SEK24)

Mutant truncated FGF1 (22-155aa) sequence, wherein, at position 55, Proline (P) was substituted for Glutamine (Q)—Q55P, and at position 62 Isoleucine (I) was substituted for Serine(S)—S62I, and at position 108 Glycine (G) was substituted for Histidine (H)—H108G; additionally, at position 153, Serine(S) was substituted with Alanine (A)—S153A. FGF1(Δ155aa; Q55P/S62I/H108G/S153A) fourfold mutation. Serial No. of the mutation: M18.

| | |
|---|---|
| Sequence No. 24 (SEK24) | 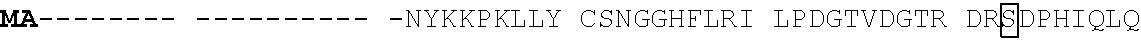 |
| FGF1 (22-155aa) | 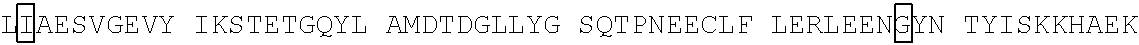 |
| FGF1_M^{(Δ155aa)} | 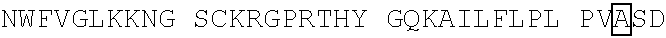 |

### Sequence No. 25 (SEK25)

Mutant full-length FGF1 (1-155aa) sequence, wherein, at position 55, Proline (P) was substituted for Glutamine (Q)—Q55P, and at position 62, Isoleucine (I) was substituted for Serine(S)—S62I, at position 108, Glycine (G) was substituted for Histidine (H)—H108G; additionally, at position 153 Serine(S) was substituted with Arginine (R)—S153R. FGF1(155aa; Q55P/S62I/H108G/S153R) fourfold mutation. Serial No. of the mutation: M19.

| | |
|---|---|
| Sequence No. 25 ( [symbol] ) | 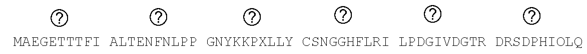 |
| FGF1 (1-155aa) | 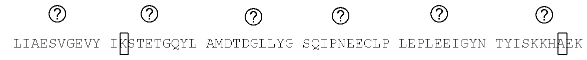 |
| FGF1—M [symbol] | 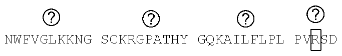 |

[symbol]

indicates data missing or illegible when filed

### Sequence No. 26 (SEK26)

Mutant truncated FGF1 (22-155aa) sequence, wherein, at position 55, Proline (P) was substituted for Glutamine (Q)—Q55P, and at position 62, Isoleucine (I) was substituted for Serine(S)—S62I, and at position 108, Glycine (G) was substituted for Histidine (H)—H108G; additionally, at position 153, Serine(S) was substituted with Arginine (R)—S153R. Fourfold mutation FGF1(Δ155aa; Q55P/S62I/H108G/S153R). Serial number of the mutation: M20.

| | |
|---|---|
| Sequence No. 26 ( [symbol] ) | 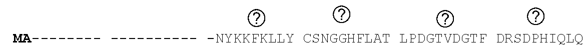 |
| FGF1 (22-155aa) | 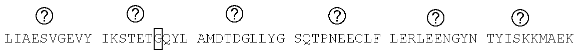 |
| FGF1_M [symbol] | 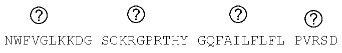 |

[symbol]

indicates data missing or illegible when filed

### Sequence No. 27 (SEK27)

Mutant full-length FGF1 (1-155aa) sequence, wherein, at position 153, Serine(S) was substituted with Aspartic Acid (D) S153D and, at position 154, Serine(S) was substituted with Aspartic Acid (D) S154D. FGF1(155aa; S153D/S154D) double mutation. Serial number of the mutation: M21.

| | |
|---|---|
| | [symbol] [symbol] [symbol] [symbol] [symbol] [symbol] |
| Sequence No. 27 ( [symbol] ) | MAFGEITTFT ALTEKFNLPP GNYKKPKLLY C [symbol] NGGHPLRI LPDGTVDGTR DRSDQHIQLQ |
| FGF1 (1-155aa) | [symbol] [symbol] [symbol] [symbol] [symbol] [symbol] |
| | LEAESVGEVY IKETETGQKL AMGTGLLYG SQTPNEECLP LEPLEIEWHYN TYISXKHARN |
| FGF_M [symbol] | 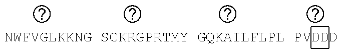 |

[symbol]

indicates data missing or illegible when filed

### Sequence No. 28 (SEK28)

Mutant truncated FGF1 (22-155aa) sequence, wherein, at position 153, Serine(S) was substituted for Aspartic Acid (D)—S153D, and at position 154 Serine(S) was substituted with Aspartic acid (D) S154D. FGF1(Δ155aa; S153D/S154D) double mutation. Serial number of the mutation: M22.

| | |
|---|---|
| | [symbol] [symbol] [symbol] [symbol] [symbol] [symbol] |
| Sequence No. 28 | MA--------- ---------- -NYKKPKLLY CSNGGNFLIRT LPDGTVDGTR DFSDQHIQLQ |
| (SEK28) | |
| FGF1 (22-155aa) | LSAKESVGEVY IKSTETCQYL AMDTDGLLYG SQTPNEECLF LERLEENHYN TYISKKHARK |
| FGF1_M [symbol] | 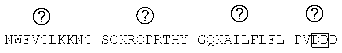 |

[symbol]

indicates data missing or illegible when filed

### Sequence No. 29 (SEK29)

Mutant full-length FGF1 (1-155aa) sequence, wherein, at position 55, Proline (P) was substituted for Glutamine (Q)—Q55P, and at position 62, Isoleucine (I) was substituted for Serine(S) S62I, at position 108, Glycine (G) was substituted for Histidine (H)—H108G; additionally, at position 153, Serine(S) was substituted with Aspartic acid (D)—S153D and at position 154 Serine(S) was substituted with Aspartic acid (D)—S154D. FGF1(155aa; Q55P/S62I/H108G/S153D/S154D) fivefold mutation. Serial No. of the mutation: M23.

| | |
|---|---|
| | [symbol] [symbol] [symbol] [symbol] [symbol] [symbol] |
| Sequence No. 29 (SEK29) | HAEGEITTFT ALTRKFNLPP GNYKKPKLLY CSNGGHPLRI LPDGTVDGTR DRSDGHIQLQ |
| | [symbol] [symbol] [symbol] [symbol] [symbol] [symbol] |
| FGF1 (1-155a) | LSAESVGEVY IKSTETGQYL AMDTDGLLYG SQYPNEECLP LEPLEEWHYN TYISKKBAEK |

| | |
|---|---|
| FGF1—M [symbol] | 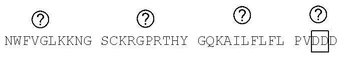 |

[symbol]

indicates data missing or illegible when filed

### Sequence No. 30 (SEK30)

Mutant truncated FGF1 (22-155aa) sequence, wherein, at position 55, Proline (P) was substituted for Glutamine (Q)—Q55P and at position 62, Isoleucine (I) was substituted for Serine(S)—S62I; at position 108, Glycine (G) was substituted for Histidine (H)—H108G; additionally, at position 153, Serine(S) was substituted with Aspartic acid (D) S153D and at position 154, Serine(S) was substituted with Aspartic Acid (D) S154D. FGF1(Δ155aa; Q55P/S62I/H108G/S153D/S154D) fivefold mutation. Serial number of the mutation: M24.

| | |
|---|---|
| Sequence No. 30 (SEK30) | 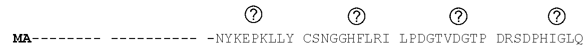 |
| FGF1 (22-155aa) | 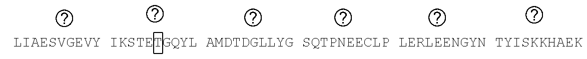 |
| FGF1_M [symbol] | 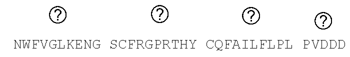 |

[symbol]

indicates data missing or illegible when filed

### Sequence No. 31 (SEK31)

Mutant full-length FGF1 (1-155aa) sequence, wherein, at position 153, Arginine (R) was substituted for Serine(S). FGF1(155aa; S153R) point mutation. Serial No. of the mutation: M25.

| | |
|---|---|
| | [symbol] [symbol] [symbol] [symbol] [symbol] [symbol] |
| Sequence No. 31 (SEK31 [symbol] | MAEGEITTFT ALTEKFNLPF GNYKKEFKLLY CBNGGHFLRI LPDGTVDGTR [symbol] DQKIQLQ |
| | 10 [symbol] [symbol] [symbol] [symbol] |
| FGF1 (1-155aa) | LSAEEVGEVY IKSTEIGQYL AMDTDGLLYG EQTPNEECLP LEKLEEZNHIYN TYLGFKAEE |
| FGF1_M [symbol] | 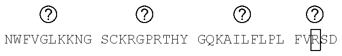 |

[symbol]

indicates data missing or illegible when filed

### Sequence No. 32 (SEK32)

Mutant full-length FGF1 (1-155aa) sequence, wherein, at position 153, Alanine (A) was substituted for Serine(S). FGF1(155aa; S153R) point mutation. Serial No. of the mutation: M26.

| | |
|---|---|
| | 10 20 30 40 50 60 |
| Sequence No. 32 (SEK32) | MAEGEITTFT ALTEKFNLPP GNYKKFPLLY CSNGGHFLRI LPDGTVDGTF DAEDQHIQLQ |
| | 70 80 90 100 119 125 |
| FGF1 (122-155aa) | LSAEKVGEVY IKETETGQKL AMDTDGLLYG EQTPNKECLP LERLEENHYN TYIEKFHAEK |
| FGF1_M [symbol] | 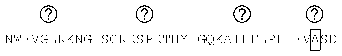 |

[symbol]

indicates data missing or illegible when filed

### Sequence No. 33 (SEK33)

Mutant truncated FGF1 (22-155aa) sequence, wherein, at position 153, Arginine (R) was substituted for Serine(S). FGF1(Δ155aa; S153R) point mutation. Serial No. of the mutation: M27.

| | |
|---|---|
| Sequence No. 33 ( [symbol] ) | MA-------- ---------- -WYKKPELLY CE [symbol] NFLPI LPDGTVDGTR DRSEQNIQLQ |
| FGF1 (22-155aa) | LEAESVGEVY IKHTETGQYL AMDTDGLLYS SQTFNEECLP LELLEENHYM TYL [symbol] KKAFK |
| FGF1_M [symbol] | 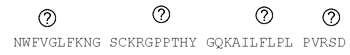 |

[symbol]

indicates data missing or illegible when filed

### Sequence No. 34 (SEK34)

Mutant truncated FGF1 (22-155aa) sequence, wherein, at position 153, Alanine (A) was substituted for Serine(S). FGF1(Δ155aa; S153A) point mutation. Serial No. of the mutation: M28.

| | |
|---|---|
| Sequence No. 34 (SER34) | MA-------- ---------- -WYFKPKLLY CENGGHFLPI LPDGTVDGTR DRSDQRIQLQ |
| FGF1 (22-155aa) | LSAKSVGEVY IKSTETGQYL AMDTDGLLYG SQTFNEECLF LERLEHNKYG TYISKKHAEK |
| FGF1_M [symbol] | 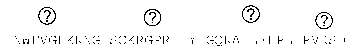 |

[symbol]

indicates data missing or illegible when filed

### Sequence No. 35 (SEK35)

Homodimer of the full-length FGF1 (1-155) mutation with the serial number M29, wherein two Sequences No. 31 were connected by the linker: GGGGSGGGGSGGGG N′ terminus-(1-155-linker-2-155)-C′ terminus. The second FGF1(155aa; S153R) monomer starts with Alanine (A) at position 170. Methionine (M) was omitted. FGF1_M29_DIMER(155aa).

| | |
|---|---|
| | [symbol] [symbol] [symbol] [symbol] [symbol] [symbol] |
| Sequence No. 35 (SEK35) | MARGEITTTFT ALTEKPNLPF GNYKKPKLLY CSNGGHPLRI LPDGTVDCTB DRSQHIQLQ |
| | [symbol] [symbol] [symbol] [symbol] [symbol] [symbol] |
| FGF1 (1-155aa) | LEANESVGEVY IKSYSTGQYL AMDTDGLLYG SQYPKEEGLP LERLEEGHYN ITTSKKHARK |
| FGF1_DIMER [symbol] | 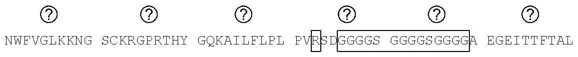 |
| | 130 140 150 160 170 180 |
| | TEKPNLPPWY KKPKELLYCSN GGMPLRILPO GTVDGTRDRS DQMIQLQLSA EEVGEVYIKS |
| | 130 140 150 160 170 180 |
| | TETGQYLAMD TDGLLYGSQT PNEECLFLEA LEENEYNTYL SKKHAEFNWF VGLKEGSCK |
| | 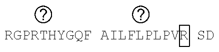 |

[symbol]

indicates data missing or illegible when filed

### Sequence No. 36 (SEK36)

Homodimer of the full-length FGF1 (1-155) mutation with the serial number M30, wherein two Sequences No. 32 were connected by the linker: GGGGSGGGGSGGGG N′ terminus-(1-155-linker-2-155)-C′ terminus. The second FGF1(155aa; S153A) monomer starts with Alanine (A) at position 170. Methionine (M) was omitted. FGF1_M30_DIMER(155aa).

| | |
|---|---|
| | [symbol] [symbol] [symbol] [symbol] [symbol] [symbol] |
| Sequence No. 36 (SEK36) | MARGEITTTFT ALTEKPNLPF GNYKKPKLLY CSNGGHPLRI LPDGTVDCTB DRSQHIQLQ |
| | [symbol] [symbol] [symbol] [symbol] [symbol] [symbol] |
| FGF1 (1-155aa) | LEANESVGEVY IKSYSTGQYL AMDTDGLLYG SQYPKEEGLP LERLEEGHYN ITTSKKHARK |
| FGF1_DIMER [symbol] | 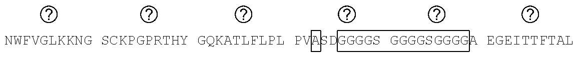 |
| | [symbol] [symbol] [symbol] [symbol] [symbol] [symbol] |
| | TEKPNLPPWY KKPKELLYCSN GGHPLRILPO GTVDGTRDRS DQMIQLQLSA EEVGEVYIKS |
| | [symbol] [symbol] [symbol] [symbol] [symbol] [symbol] |
| | TETGQYLAMD TDGLLKGSGT PNEECLFLEA LEENEYNTYL SKKHAEFNWF VGLKEGSCK |
| | 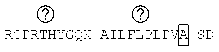 |

[symbol]

indicates data missing or illegible when filed

### Sequence No. 37 (SEK37)

Homodimer of the truncated FGF1 (2-155), wherein two Sequences No. 33 were connected by the linker: GGGGSGGGGSGGGG N′ terminus-(22-155-linker-22-155)-C′ terminus. In the sequence, the bold font indicates the first 2 amino acids of the first FGF1(Δ155aa; S153R) monomer and the first amino acid of the second FGF1(Δ155aa; S153R) monomer, the presence of which results from the method of cloning of the truncated FGF1(aa 22-155) sequence into an expression vector. Methionine (M) is encoded by the start codon AUG from which the protein expression process starts and the presence of which is required at N′ terminus. Alanine (A) was added to the sequence as a result of adding two nucleotides to the sequence flanking the restriction site in order to preserve an appropriate reading frame in the transcription process. The addition of alanine does not cause any physical and chemical changes of the protein due to its non-polar and aliphatic nature. The second FGF1(Δ155aa; S153R) monomer starts with Alanine (A) at position 170. Methionine (M) was omitted. FGF1_M31_DIMER(Δ155aa).

| | |
|---|---|
| Sequence No. 37 ( [symbol] ) | MA-------- ---------- - [symbol] NYNYKKPALLY CEUGGFLEI LPGGTVDGTE DRSCQHTQIQ |
| FGP1 (22-155aa) | LAEASVGEVY IKETETGQKL AMDTDGLLYG SQTPNEECLF LERLEENKVW TYISKKHAEK |
| FGF1_DIMER [symbol] | 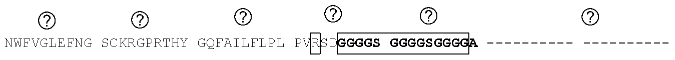 |
| | ------NY KKPELLYCSW [symbol] GVDGTFDRS SQHIQLQLSA ESVSEVIIKS |
| | TETGQVLAMD TDGLLYSSQT HNEECLFLER LEEDKYVWTYI SEKFHAEKINF VGLKKGDCK |
| | 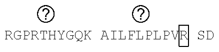 |

[symbol]

indicates data missing or illegible when filed

### Sequence No. 38 (SEK38)

Homodimer of the truncated FGF1 (2-155), wherein two Sequences No. 34 were connected by the linker: GGGGSGGGGSGGGG N′ terminus-(22-155-linker-22-155)-C′ terminus. In the sequence, the bold font indicates the first 2 amino acids of the first FGF1(Δ155aa; S153A) monomer and the first amino acid of the second FGF1(Δ155aa; S153A) monomer, the presence of which results from the method of cloning of the truncated FGF1(aa 22-155) sequence into an expression vector. Methionine (M) is encoded by the start codon AUG from which the protein expression process starts and the presence of which is required at N′ terminus. Alanine (A) was added to the sequence as a result of adding two nucleotides to the sequence flanking the restriction site in order to preserve an appropriate reading frame in the transcription process. The addition of alanine does not cause any physical and chemical changes of the protein due to its nonpolar and aliphatic nature. The second FGF1(Δ155aa; S15A) monomer starts with Alanine (A) at position 170. Methionine (M) was omitted. FGF1_M32_DIMER(Δ155aa).

| | |
|---|---|
| Sequence No. 37 ( [symbol] ) | MA-------- ---------- -NYNYKKPALLY CEUGGFLEI LPGGTVDGTE DRSCQHTQIQ |
| FGP1 (22-155aa) | LAEASVGEVY IKETETGQKL AMDTDGLLYG SQTPNEECLF LERLEENKVW TYISKKHAEK |
| FGF1_DIMER [symbol] | 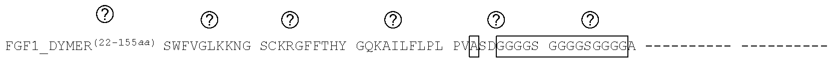 |
| | ------NY KKPELLYCSW [symbol] GVDGTFDRS SQHIQLQLSA ESVSEVIIKS |
| | TETGQVLAMD TDGLLYSSQT HNEECLFLER LEEDKYVWTYI SEKFHAEKINF VGLKKGDCK |
| | 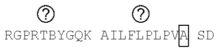 |

[symbol]

indicates data missing or illegible when filed

### BRIEF DESCRIPTION OF THE FIGURES

FIG. 1 shows a proliferation assay measured by MTT method using the commercial CellTiter 96® Non-Radioactive Cell Proliferation Assay kit (Promega). NIH3T3 cells were seeded at 2000 cell/well on a 96-well plate using Dulbecco's Modified Eagle's Medium (DMEM) supplemented with 10% calf serum (CS). On the next day the cells were starved for 5 hours in DMEM without CS; subsequently, proteins were added at the specified concentrations, and were incubated for 48 hours. The MTT assay was performed in accordance with the manufacturer's protocol. The results of the experiment showed an increase in proliferation of both forms of FGF1 without significant differences between the truncated form—FGF1 protein (Δ155aa) and the full-length form—FGF1(155aa) protein.

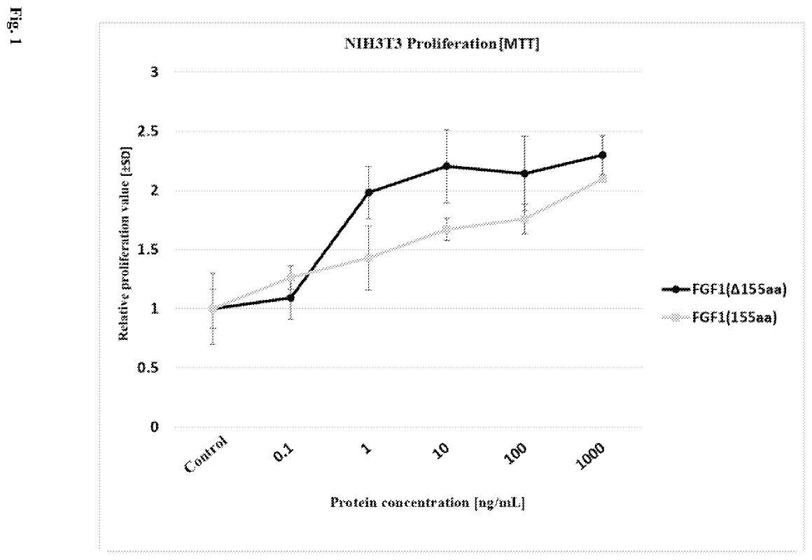

FIG. 2 shows the results of a proliferation assay measured by MTT method using the commercial CellTiter 96® Non-Radioactive Cell Proliferation Assay kit (Promega). NIH3T3 cells were seeded at 2000 cells/well on a 96-well plate using DMEM supplemented with 10% CS. On the following day the cells were starved for 5 hours in DMEM without CS; subsequently, proteins were added at the specified concentrations, and were incubated for 48 hours. The MTT assay was performed according to the manufacturer's protocol. The results of the experiment showed a significant induction of proliferation by the full-length form of the wild-type FGF1 protein (155aa) and insignificant induction of proliferation by S114A mutant (with reduced affinity to CK2), both in the truncated form—FGF1(Δ155aa; S114A) and the full-length form—FGF1(155aa; S114A).

FIG. 3 shows the results of a proliferation assay measured by MTT method using the commercial CellTiter 96® Non-Radioactive Cell Proliferation Assay kit (Promega). NIH3T3 cells were seeded at 2000 cells/well on a 96-well plate using DMEM supplemented with 10% CS. On the following day the cells were starved for 5 hours in DMEM without CS; subsequently, proteins were added at the specified amounts, and were incubated for 48 hours. The MTT assay was performed according to the manufacturer's protocol. The results of the experiment showed a significant induction of proliferation by the full form of the wild-type FGF1 protein (155aa) and insignificant induction of proliferation by L150D mutant (with reduced affinity to the FGFR1 receptor), both in the truncated form—FGF1(Δ155aa; L150D) and the full-length form—FGF1(155aa; L150D).

FIG. 4 shows the results of a proliferation assay measured by MTT method using the commercial CellTiter 96® Non-Radioactive Cell Proliferation Assay kit (Promega). NIH3T3 cells were seeded at 2000 cells/well on a 96-well plate using DMEM supplemented with 10% CS. On the following day the cells were starved for 5 hours in DMEM without CS; subsequently, proteins were added at the specified concentrations, and were incubated for 48 hours. The MTT assay was performed according to the manufacturer's protocol. The results of the experiment showed a significant induction of proliferation by the full form of the wild-type FGF1 protein (155aa) and insignificant induction of proliferation by S114A/L150D mutant (with reduced affinity to CK2 and the FGFR1 receptor), both in the truncated form—FGF1(Δ155aa; S114A/L150D) and the full-length form—FGF1(155aa; S114A/L150D).

FIG. 5 shows the results of a proliferation assay measured by MTT method using the commercial CellTiter 96® Non-Radioactive Cell Proliferation Assay kit (Promega). NIH3T3 cells were seeded at 2000 cells/well on a 96-well plate using DMEM supplemented with 10% CS. On the next day the cell were starved for 5 hours in DMEM without CS; subsequently, proteins were added in the specified concentrations, and were incubated for 48 hours. The MTT assay was performed according to the manufacturer's protocol. The results of the experiment showed a significant induction of proliferation by the wild-type form of FGF1 protein (155aa) and insignificant induction of proliferation by Q55P/S62I/H108G/S114A/L150D mutant (the mutant with increased thermal stability and reduced affinity to CK2 and the FGFR1 receptor), both in the truncated form—FGF1(Δ155aa; Q55P/S62I/H108G/S114A/L150D), and the full-length form—FGF1(155aa; Q55P/S62I/H108G/S114A/L150D).

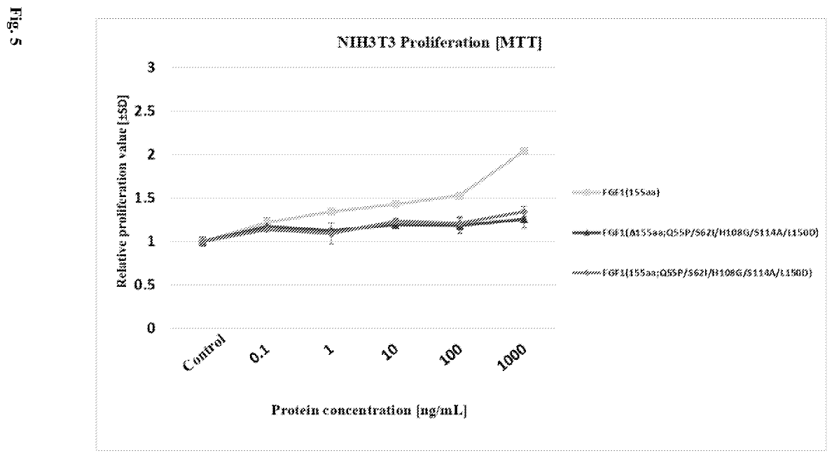

FIG. 6 shows the results of a proliferation assay measured by MTT method using the commercial CellTiter 96® Non-Radioactive Cell Proliferation Assay kit (Promega). NIH3T3 cells were seeded at 2000 cells/well on a 96-well plate using DMEM supplemented with 10% CS. On the following day the cells were starved for 5 hours in DMEM without CS; subsequently, proteins were added at the specified concentrations, and were incubated for 48 hours. The MTT assay was performed according to the manufacturer's protocol. The proteins used in the experiment are mutants of the full-length FGF1(155aa) protein. The results of the experiment showed a very high induction of proliferation by the mutant with increased thermal stability-FGF1(155aa; Q55P/S62I/H108G). Moreover, the experiment showed an induction of proliferation by the wild-type form of FGF1(155aa) protein and insignificant induction of proliferation by mutants: FGF1(155aa; Q55P/S62I/H108G/S114A/L150D), FGF1(155aa; S114A), FGF1(155aa; L150D) and FGF1(155aa; S114A/L150D).

FIG. 7 shows the results of a proliferation assay measured by MTT method using the commercial CellTiter 96® Non-Radioactive Cell Proliferation Assay kit (Promega). NIH3T3 cells were seeded at 2000 cells/well on a 96-well plate using DMEM supplemented with 10% CS. On the following day the cells were starved for 5 hours in DMEM without CS; subsequently, proteins were added at the specified concentrations, and were incubated for 48 hours. The MTT test was performed according to the manufacturer's protocol. The proteins used in the experiment are mutants of the full-length form of the FGF1(155aa) protein. The results of the experiment showed a very high induction of proliferation by the mutants with increased thermal stability-FGF1(155aa; Q55P/S62I/H108G), FGF1(155aa; Q55P/S62I/H108G/S153A), FGF1(155aa; Q55P/S62I/H108G/S153R). Moreover, the experiment showed an induction of proliferation by the wild-type form of FGF1(155aa) protein, and mutants: FGF1(155aa; S153A), FGF1(155aa; S153R). FGF1(155aa; Q55P/S62I/H108G/S114A/L150D) mutant showed an insignificant induction of proliferation.

FIG. 8 shows the results of a proliferation assay measured by MTT method using the commercial CellTiter 96® Non-Radioactive Cell Proliferation Assay kit (Promega). NIH3T3 cells were seeded at 2000 cells/well on a 96-well plate using DMEM supplemented with 10% CS. On the following day the cells were starved for 5 hours in DMEM without CS; subsequently, proteins were added at the specified concentrations, and were incubated for 48 hours. The MTT assay was performed according to the manufacturer's protocol. The proteins used in the experiment are mutants of the full-length form of the FGF1(155aa) protein. The results of the experiment showed a very high induction of proliferation by the mutants: FGF1(155aa; S153R) and FGF1(155aa; Q55P/S62I/H108G/S114A/L150D). Moreover, the experiment showed an induction of proliferation by the wild-type form of the FGF1(155aa) protein and mutants: FGF1(155aa; S153A), FGF1(155aa; Q55P/S62I/H108G). FGF1(155aa; Q55P/S62I/H108G/S114A/L150D) mutant showed an insignificant induction of proliferation.

FIG. 9 shows the results of a proliferation assay measured by MIT method using the commercial CellTiter 96® Non-Radioactive Cell Proliferation Assay kit (Promega). NIH3T3 cells were seeded at 2000 cells/well on a 96-well plate with the use of Dulbecco's Modified Eagle's Medium (DMEM) with 10% addition of CS. On the following day the cells were starved for 5 hours in DMEM without CS; subsequently, proteins were added at the specified concentrations, and were incubated for 48 hours. The MTT assay was performed according to the manufacturer's protocol. The results of the experiment showed an increase in the proliferation of both forms of FGF1 without significant differences between the truncated form—FGF1(Δ155aa) protein and the truncated dimeric form—FGF1_DIMER(Δ155aa) protein.

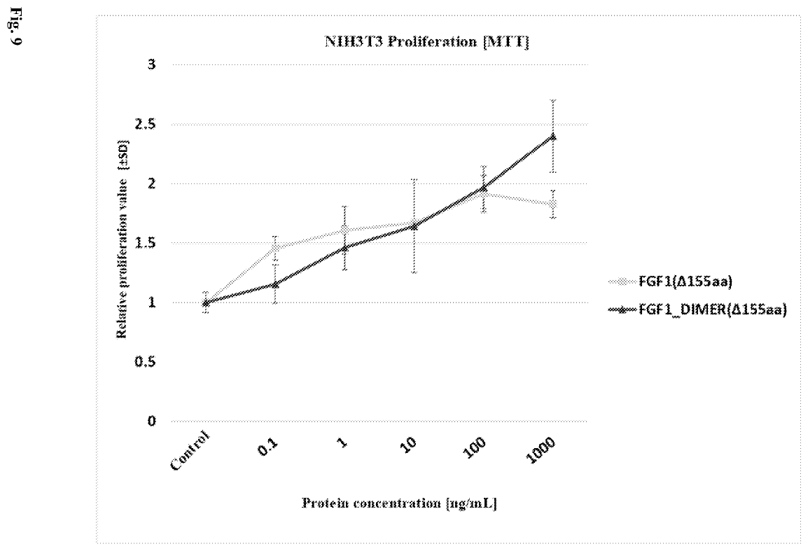

FIG. 10 shows the results of a proliferation assay measured by MTT method using the commercial CellTiter 96® Non-Radioactive Cell Proliferation Assay kit (Promega). NIH3T3 cells were seeded at 2000 cells/well on a 96-well plate using DMEM supplemented with 10% CS. On the following day the cells were starved for 5 hours in DMEM without CS; subsequently, proteins were added at the specified concentrations (with an addition of heparin, 10 U/ml, or without it), and were incubated for 48 hours. The MTT assay was performed according to the manufacturer's protocol. The results of the experiment showed a positive effect of heparin on the increase in proliferation for the wild-type form of the FGF1(Δ155aa) protein. The lack of heparin had an effect on the intensity of proliferation but did not stop it. The results unambiguously indicate that heparin does not influence the effectiveness of proliferation induction by FGF1(Δ155aa). Moreover, the experiment showed an insignificant induction of proliferation by FGF1(Δ155aa; Q55P/S62I/H108G/S114A/L150D) mutant, both with or without the addition of heparin.

FIG. 11 shows the results of a glucose uptake assay measured by the chemiluminescence method using the commercial Glucose Uptake-Glo™ Assay kit (Promega). In the test, adipocytes differentiated from mouse fibroblasts 3T3-L1 were used according to the protocol provided by the manufacturer, Glucose Uptake-Glo (Promega). The differentiated adipocytes were seeded at 50000 cells/well on a 96-well Poly-D-Lysine coated plate using DMEM supplemented with 10% foetal bovine serum (FBS). On the following day, proteins were added at the specified concentrations in DMEM without FBS, and were incubated for 16 hours. The glucose uptake assay was performed according to the manufacturer's protocol. The results of the experiment showed an increase in glucose uptake for both forms of FGF1, without significant differences between the truncated form—FGF1(Δ155aa) protein and the full-length FGF1(155aa) protein.

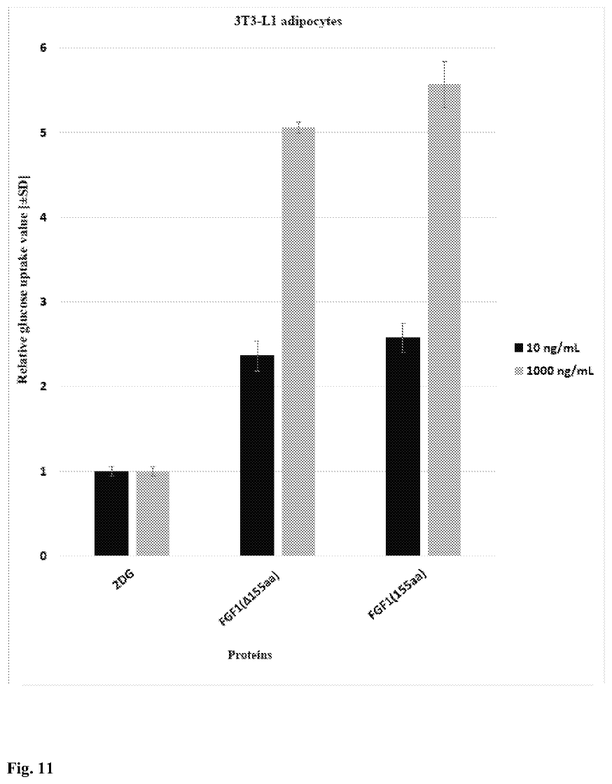

FIG. 12 shows the results of a glucose uptake assay measured by the chemiluminescence method using the commercial Glucose Uptake-Glo™ Assay kit (Promega). In the test, adipocytes differentiated from mouse fibroblasts 3T3-L1 were used according to the protocol provided by the manufacturer, Glucose Uptake-Glo (Promega). The differentiated adipocytes were seeded at 50000 cells/well on a 96-well Poly-D-Lysine coated plate using DMEM supplemented with 10% FBS. On the following day, proteins were added at the specified concentrations in DMEM without FBS, and were incubated for 16 hours. The glucose uptake assay was performed according to the manufacturer's protocol. The results of the experiment showed an increase in glucose uptake for the wild-type form of the FGF1(155aa) protein and a significant increase in glucose uptake (exceeding the action of FGF1(155aa)) for S114A mutant, both in the truncated form—FGF1(Δ155aa; S114A) and the full-length form—FGF1(155aa; S114A).

FIG. 13 shows the results of a glucose uptake assay measured by the chemiluminescence method using the commercial Glucose Uptake-Glo™ Assay kit (Promega). In the test, adipocytes differentiated from mouse fibroblasts 3T3-L1 were used according to the protocol provided by the manufacturer, Glucose Uptake-Glo (Promega). The differentiated adipocytes were seeded at 50000 cells/well on a 96-well Poly-D-Lysine coated plate using DMEM supplemented with 10% FBS. On the following day, proteins were added at the specified concentrations in DMEM without FBS, and were incubated for 16 hours. The glucose uptake assay was performed according to the manufacturer's protocol. The results of the experiment showed an increase in glucose uptake for the wild-type form of the FGF1(155aa) protein and a significant decrease in glucose uptake (compared to FGF1(155aa)) for L150D mutant, both in the truncated form—FGF1(Δ155aa; L150D) and the full-length form—FGF1(155aa; L150D).

FIG. 14 shows the results of a glucose uptake assay measured by the chemiluminescence method using the commercial Glucose Uptake-Glo™ Assay kit (Promega). In the test, adipocytes differentiated from mouse fibroblasts 3T3-L1 were used according to the protocol provided by the manufacturer, Glucose Uptake-Glo (Promega). The differentiated adipocytes were seeded at 50000 cells/well on a 96-well Poly-D-Lysine coated plate using DMEM supplemented with 10% FBS. On the following day, proteins were added at the specified concentrations in DMEM without FBS, and were incubated for 16 hours. The glucose uptake assay was performed according to the manufacturer's protocol. The results of the experiment showed an increase in glucose uptake for the wild-type form of the FGF1(155aa) protein and a significant decrease in glucose uptake (compared to the FGF1(155aa)) for S114A/L150D mutant, both in the truncated form—FGF1(Δ155aa; S114A/L150D), and the full-length form—FGF1(155aa; S114A/L150D).

FIG. 15 shows the results of a glucose uptake assay measured by the chemiluminescence method using the commercial Glucose Uptake-Glo™ Assay kit (Promega). In the test, adipocytes differentiated from mouse fibroblasts 3T3-L1 were used according to the protocol provided by the manufacturer, Glucose Uptake-Glo (Promega). The differentiated adipocytes were seeded at 50000 cells/well on a 96-well Poly-D-Lysine coated plate using DMEM supplemented with 10% FBS. On the following day, proteins were added at the specified concentrations in DMEM without FBS, and were incubated for 16 hours. The glucose uptake assay was performed according to the manufacturer's protocol. The results of the experiment showed a significant increase in glucose uptake for the wild-type form of the FGF1(155aa) protein and an increase in glucose uptake for the 1000 ng/ml dose of Q55P/S62I/H108G/S114A/L150D mutant, both in the truncated form—FGF1(Δ155aa; Q55P/S62I/H108G/S114A/L150D) and the full-length form—FGF1(155aa; Q55P/S62I/H108G/S114A/L150D).

FIG. 16 shows the results of a glucose uptake assay measured by the chemiluminescence method using the commercial Glucose Uptake-Glo™ Assay kit (Promega). In the test, adipocytes differentiated from mouse fibroblasts 3T3-L1 were used according to the protocol provided by the manufacturer, Glucose Uptake-Glo (Promega). The differentiated adipocytes were seeded at 50000 cells/well on a 96-well Poly-D-Lysine coated plate using DMEM supplemented with 10% FBS. On the following day, proteins were added at the specified concentrations in DMEM without FBS and were incubated for 16 hours. The glucose uptake assay was performed according to the manufacturer's protocol. The proteins used in the experiments are mutants of the full-length FGF1(155aa) protein. The results of the experiment showed a very high glucose uptake for the mutant with increased thermal stability-FGF1(155aa; Q55P/S62I/H108G). Moreover, it showed an increased glucose uptake for FGF1(155aa), FGF1(155aa; Q55P/S62I/H108G/S114A/L150D and FGF1(155aa; S114A). FGF1(155aa; L150D) and FGF1(155aa; S114A/L150D) exhibited an insignificant or no increase in glucose uptake.

FIG. 17 shows the results of a glucose uptake assay measured by the chemiluminescence method using the commercial Glucose Uptake-Glo™ Assay kit (Promega). In the test, adipocytes differentiated from mouse fibroblasts 3T3-L1 were used according to the protocol provided by the manufacturer, Glucose Uptake-Glo (Promega). The differentiated adipocytes were seeded at 50000 cells/well on a 96-well Poly-D-Lysine coated plate using DMEM supplemented with 10% FBS. On the following day, proteins were added at the specified concentrations in DMEM without FBS, and were incubated for 16 hours. The glucose uptake assay was performed according to the manufacturer's protocol. The proteins used in the experiments are mutants of the full-length FGF1(155aa) protein. The results of the experiment showed a very high induction of glucose uptake for the mutants with increased thermal stability FGF1(155aa; Q55P/S62I/H108G), FGF1(155aa; Q55P/S62I/H108G/S153A), FGF1(155aa; Q55P/S62I/H108G/S153R). Moreover, the experiment showed an increased glucose uptake for FGF1(155aa) and FGF1(155aa; Q55P/S62I/H108G/S114A/L150D), whereas FGF1(155aa; Q55P/S62I/H108G/S153A) and FGF1(155aa; Q55P/S62I/H108G/S153R) showed the highest induction of glucose uptake.

FIG. 18 shows the results of a glucose uptake assay measured by the chemiluminescence method using the commercial Glucose Uptake-Glo™ Assay kit (Promega). In the test, adipocytes differentiated from mouse fibroblasts 3T3-L1 according to the protocol provided by the manufacturer, Glucose Uptake-Glo (Promega). The differentiated adipocytes were seeded at 50000 cells/well on a 96-well Poly-D-Lysine coated plate using DMEM supplemented with 10% FBS. On the following day, proteins were added at the specified concentrations in DMEM without FBS, and were incubated for 16 hours. The glucose uptake assay was performed according to the manufacturer's protocol. The results of the experiment showed an increase in glucose uptake for both dimeric forms of FGF1, without significant differences between the truncated form of the FGF1_DIMER(Δ155aa) protein and the full-length form of the FGF1_DIMER(155aa) protein.

FIG. 19 shows the results of an analysis of protein expression and phosphorylation of proteins of FGF:FGFR dependent pathway performed by Western Blot (WB) method with membrane scanning performed by the chemiluminescence method using ChemiDock (Bio-Rad) equipment. In the test, adipocytes differentiated from mouse fibroblasts 3T3-L1 according to the protocol provided by the manufacturer, Glucose Uptake-Glo (Promega). The differentiated adipocytes were seeded at 50000 cells/well on a 96-well Poly-D-Lysine coated plate using DMEM supplemented with 10% FBS. On the following day, the cells were starved for 5 hours in DMEM without FBS; subsequently proteins were added at the concentration of 100 ng/ml, and were incubated for 10 minutes to study phosphorylation of proteins of FGF:FGFR-dependent pathway, and for 16 hours to study the expression of protein Glut1. After incubation the proteins were washed with cold PBS, and cell lysis was performed using a RIPA buffer with an addition of protease and phosphatase inhibitors. The proteins were separated by SDS-PAGE electrophoresis and were transferred onto a nitrocellulose membrane. Blocked membranes were incubated for 16 hours at the temperature of 4° with primary antibodies in 1:1000 dilution. Anti-rabbit-HRP secondary antibodies were incubated for 1 hour at room temperature. Clarity Max ECL buffers were used to obtain a chemiluminescent signal. The control of protein concentration was performed by means of Stain-Free signal analysis. The results of the experiment showed an increase in the phosphorylation of the FGFR1 receptor and the FGFR1 receptor dependent proteins, i.e. pErk1/2 and pFRS2a for FGF1(155aa), FGF1(155aa; Q55P/S62I/H108G) and FGF1(155aa; S114A). FGF1(155aa; Q55P/S62I/H108G/S114A/L150D insignificantly activated FGF1:FGFR pathway whereas FGF1(155aa; L150D) and FGF1(155aa; S114A/L150D) showed lack of activation.

FIG. 20 shows the results of the in vivo study of the blood glucose concentration reducing effect in a diabetic strain of db/db mice (BKS.Cg−+Leprdb/+Leprdb/OlaHsd) after administration of 0.5 mg/kg m.c. of the wild-type FGF1(Δ155aa) protein with and without the addition of 10 U/ml heparin. The control group was db/db mice receiving a carrier. The results unambiguously indicate a positive effect of FGF1(Δ155aa) on reducing glucose to normoglycemia and lack of heparin action on the effectiveness of glucose reduction in db/db mice.

FIG. 21 shows the results of the in vivo study of the blood glucose concentration reducing effect in a diabetic strain of db/db mice (BKS.Cg−+Leprdb/+Leprdb/OlaHsd) after administration of 1 mg/kg m.c. of wild-type FGF1 protein (Δ155aa) and FGF1(Δ155aa; S114A) mutant. The control group was db/db mice receiving a carrier. The results show a positive effect of FGF1(Δ155aa) and FGF1(Δ155aa; S114A) mutant on reducing glucose to normoglycemia in db/db mice.

FIG. 22 shows the results of the in vivo study of the blood glucose concentration reducing effect in a diabetic strain of db/db mice (BKS.Cg−+Leprdb/+Leprdb/OlaHsd) after administration of 1 mg/kg m.c. of the wild-type FGF1 protein (Δ155aa) and FGF1(Δ155aa; L150D) mutant. The control group was db/db mice receiving a carrier. The results indicate a positive effect of FGF1(Δ155aa) on reducing glucose to normoglycemia in db/db mice and lack of effective action of FGF1(Δ155aa; L150D) mutant.

FIG. 23 shows the results of the in vivo study of the blood glucose concentration reducing effect in a diabetic strain of db/db mice (BKS.Cg−+Leprdb/+Leprdb/OlaHsd) after administration of 1 mg/kg m.c. of the FGF1 protein (Δ155aa) and FGF1(Δ155aa; Q55P/S62I/H108G) mutant. The control group was db/db mice receiving a carrier. The results indicate a positive effect of FGF1(Δ155aa) and FGF1(Δ155aa; Q55P/S62I/H108G) on reducing glucose to normoglycemia in db/db mice, with the effectiveness of action of FGF1(Δ155aa; Q55P/S62I/H108G) mutant being higher compared to FGF1(Δ155aa).

FIG. 24 shows the results of the in vivo study of the blood glucose concentration reducing effect in a diabetic strain of db/db mice (BKS.Cg−+Leprdb/+Leprdb/OlaHsd) after administration of 1 mg/kg m.c. of the FGF1(Δ155aa) protein and Q55P/S62I/H108G/S114A/L150D mutants. The control group was db/db mice receiving a carrier. The results indicate a positive effect of FGF1(Δ155aa) and Q55P/S62I/H108G/S114A/L150D mutants on reducing glucose to normoglycemia in db/db mice without significant differences between the truncated form—FGF1(Δ155aa; Q55P/S62I/H108G/S114A/L150D) and the full-length form—FGF1(155aa; Q55P/S62I/H108G/S114A/L150D).

FIG. 25 shows the results of the in vivo study of the blood glucose concentration reducing effect in a diabetic strain of db/db mice (BKS.Cg−+Leprdb/+Leprdb/OlaHsd) after administration of 1 mg/kg m.c. of the FGF1 protein (Δ155aa), FGF1(155aa) and dimeric form of the truncated mutein-FGF1_DIMER(Δ155aa). The control group was db/db mice receiving a carrier. The results indicate a positive effect of FGF1(Δ155aa) and FGF1(155aa) on reducing glucose to normoglycemia in db/db mice and lack of effective action of the truncated dimeric form—FGF1_DIMER(Δ155aa).

FIG. 26 shows the results of the in vivo study of the blood glucose concentration reducing effect in a diabetic strain of mice db/db (BKS.Cg−+Leprdb/+Leprdb/OlaHsd) after administration of 5 mg/kg m.c. of FGF1 protein (Δ155aa), mutant FGF1(155aa; Q55P/S62I/H108G/S114A/L150D) and dimeric form FGF1_M8_DIMER(155aa). The control group was db/db mice receiving a carrier. The results indicate a positive effect of FGF1(Δ155aa) and FGF1(155aa; Q55P/S62I/H108G/S114A/L150D) on reducing glucose to normoglycemia in db/db mice and a lower, compared to the monomeric version, effectiveness of action of the dimeric form—FGF1_M8_DIMER(155aa).

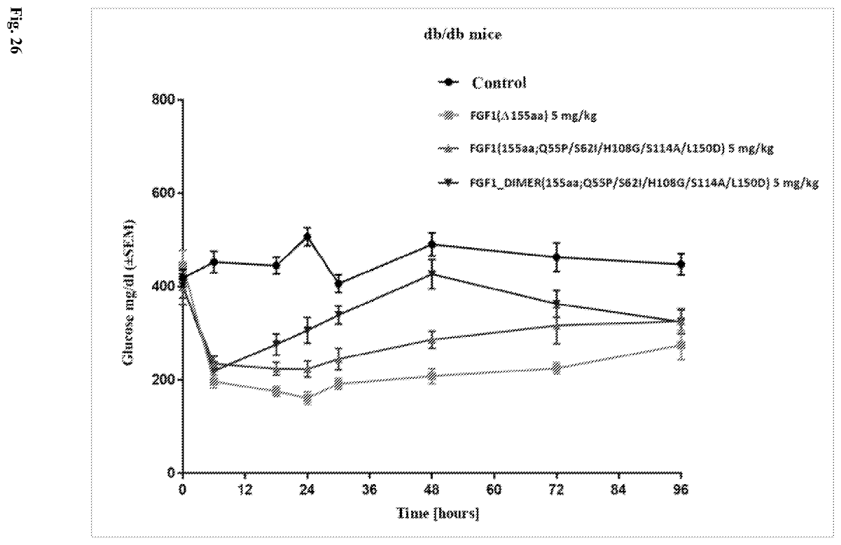

FIG. 27 shows the results of the in vivo study of the blood glucose concentration reducing effect in a diabetic strain of db/db mice (BKS.Cg−+Leprdb/+Leprdb/OlaHsd) after administration of 1 mg/kg m.c. of the FGF1(Δ155aa) protein, and three doses: 1, 2.5, 5 mg/kg m.c. for FGF1(155aa; Q55P/S62I/H108G/S114A/L150D). The control group was db/db mice receiving a carrier. The results indicate a positive effect of FGF1(Δ155aa) and FGF1(155aa; Q55P/S62I/H108G/S114A/L150D) on reducing glucose to normoglycemia in db/db mice with clear dose-dependence for FGF1(155aa; Q55P/S62I/H108G/S114A/L150D).

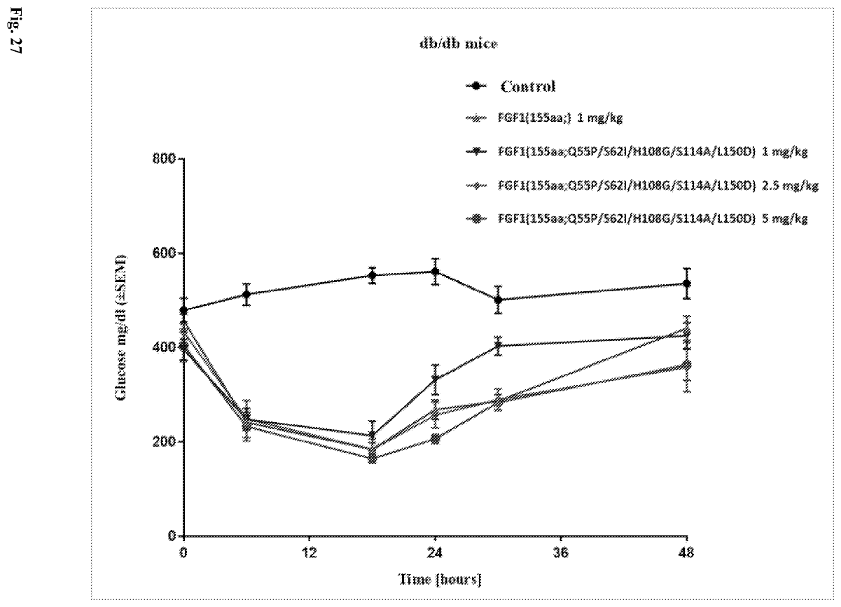

### EXAMPLES

All the procedures, assays and experimental analyses described below were performed with the use of commercially available test kits, reactants and apparatus, following recommendations of the manufacturers of the applied kits, reagents and apparatus, unless otherwise clearly indicated herein; the inventors used standard, commonly known methods applied in the field to which the present invention belongs.

### Example 1

Production of constructs encoding FGF1 protein variants according to the invention and disclosure, and their expression and purification.

Wild-type variant FGF1 WT—cDNA (GenBank accession number NM 001354952.2, 468 bp, encoding human fibroblast growth factor 1 (FGF-1), was optimized to an expression in E. coli cells and a gene flanked with the restriction sites for enzymes: Ndel at 5′ terminus and XhoI at 3′ terminus (Gene Synthesis, Thermo Fisher Scientific) was synthetized. The synthetic gene was cloned into a modified expression vector pCPBT0010 prepared by Celon Pharma, using the above specified restriction enzymes. The designed construct for FGF1(155aa) protein did not have labels facilitating purification and additional amino acids at N or C terminuses.

All muteins constructed in accordance with the invention, i.e. mutated sequences of proteins FGF1(Δ155aa) (short variants) and FGF1(155aa) (full-length variants) comprising one, two, three, four of five point mutations were obtained in a standard way in a PCR reaction, unless indicated otherwise, in accordance with the methodology of directed mutagenesis and then transformation to DH5 E. coli, in accordance with the methodology described by Hanahan et al. (Hanahan, D., Jessee, J., & Bloom, F. R. (1991). Plasmid transformation of Escherichia coli and other bacteria. Methods in Enzymology, 204, 63-113. https://doi.org/10.1016/0076-6879(91)04006-a).

Construction of muteins: FGF1(155aa; L150D), FGF1(155aa; S114A)—single mutants were prepared on the basis of the FGF1 sequence (1-155 aa, Sequence No. 1) in a PCR reaction in accordance with the methodology of Site-Directed Point Mutagenesis described by Stratagene. All amplification reactions (50 μl) comprised a polymerase buffer, dNTP, template DNA, primers complementary to sense and antisense strands carrying a mutated codon and Q5 Hot Start High-Fidelity polymerase (New England Biolab). Reaction mixture was transformed to DH5 E. coli cells in accordance with the Hanahan's methodology. (Hanahan et al., 1991).

Construction of mutein: FGF1(155aa; S114A/L150D), FGF1(155aa; Q55P/S62I/H108G/S114A/L150D)—a mutein with two point mutations was prepared by modifying cDNA codons of the gene for the wild-type FGF1(aa 1-155) protein. DNA sequences were optimized for an expression in E. coli cells, genes were synthesized and prepared in accordance with the above description.

Construction of mutein: FGF1(155aa; Q55P/S62I/H108G/S114A/L150D)—A multiple mutein was prepared by modifying cDNA codons of the gene for the wild-type FGF1(aa 1-155) protein (Sequence nr 1). DNA sequences were optimized for an expression in E. coli cells, genes were synthesized and prepared in accordance with the above description.

Construction of muteins: FGF1(155aa; S153A), FGF1(155aa; S153R)—single mutants were prepared on the basis of the FGF1 sequence (1-155 aa, Sequence No. 1) in a PCR reaction in accordance with Site-Directed Point Mutagenesis described by Stratagene. All amplification reactions (50 μl) comprised a polymerase buffer, dNTP, template DNA, primers complementary to sense and antisense strands carrying a mutated codon and Q5 Hot Start High-Fidelity polymerase (New England Biolab). Reaction mixture was transformer to DH5 E. coli cells in accordance with the Hanahan's methodology (Hanahan et al., 1991).

Construction of muteins: FGF1(155aa; Q55P/S62I/H108G/S153A); FGF1(155aa; Q55P/S62I/H108G/S153R)—Multiple muteins were prepared by modifying cDNA codons of the gene for the wild-type FGF1(aa 1-155) protein (Sequence No. 1). DNA sequences were optimized for an expression in E. coli cells, genes were synthesized and prepared in accordance with the above description.

Construction of mutein: FGF1(155aa; S153D/S154D)—a single mutant was prepared on the basis of the FGF1 sequence (1-155 aa, Sequence nr 1) in a PCR reaction in accordance with the methodology of Site-Directed Point Mutagenesis described by Stratagene. All amplification reactions (50 μl) comprised a polymerase buffer, dNTP, template DNA, primers complementary to sense and antisense strands carrying a mutated codon and Q5 Hot Start High-Fidelity polymerase (New England Biolab). Reaction mixture was transformed to DH5 E. coli cells in accordance with the Hanahan's methodology (Hanahan et al., 1991).

Construction of mutein: FGF1(155aa; Q55P/S62I/H108G/S153D/S154D)—multiple muteins were prepared by modifying cDNA codons of the gene for the wild-type FGF1(aa 1-155) protein (Sequence nr 1). DNA sequences were optimized for an expression in E. coli cells, genes were synthesized and prepared in accordance with the above description.

Construction of muteins: FGF1(Δ155aa; Q55P/S62I/H108G/S153D/S154D); FGF1(Δ155aa; S114A); FGF1(Δ155aa; L150D); FGF1(Δ155aa; S114A/L150D); FGF1(Δ155aa; Q55P/S62I/H108G/S153A); FGF1(Δ155aa; Q55P/S62I/H108G/S153R); FGF1(Δ155aa; S153A), FGF1(Δ155aa; S153R)—constructs for these mutated proteins included cDNA encoding information for the wild-type form of the FGF1 protein truncated at N-terminus and its truncated muteins. DNA sequences were optimized for an expression in E. coli cells, synthetic genes were ordered and prepared in accordance with the above description.

### Expression and Purification of FGF1 Protein Variants According to the Invention and Disclosure

FGF1 protein and its variants in the long FGF1(155aa) version and truncated FGF1(Δ155aa) version were expressed in E. coli cells in TB (Terrific Broth; Sigma) medium supplemented with antibiotics. Kanamycin comprising TB medium was inoculated with an overnight pre-culture. Protein expression was induced with IPTG and then the cultures were grown for 20 h. Bacterial cultures were centrifuged for 15 min. at 6.000×g at 4° C. The pellets obtained were suspended in a lysis buffer and were incubated for 30 min, and then they were subjected to sonication on ice for 5 min. Filtered soluble fractions (supernatant) obtained by centrifuging the samples for 30 min at 20.000×g at 4° C. were loaded on a heparin column (Heparin 6 FastFlow, Cytiva). Resin-unbound proteins were washed out with buffer A and FGF1 elution was performed using a gradient in buffer B. Eluted FGF1 proteins were subjected to a further process of gel filtration (HiLoad 16/600 Superdex 75 prep grade column, Cytivia). The proteins produced were subjected to a quantitative analysis (A280) using extinction coefficient and qualitative analyses: purity using SDS/PAGE, capillary electrophoresis, thermal shift, SEC-HPLC, MS. The proteins were aliquoted, frozen in liquid nitrogen and stored at −80° C.

### Example 2

Confirming the effectiveness of the exogenous recombinant human FGF1 protein.

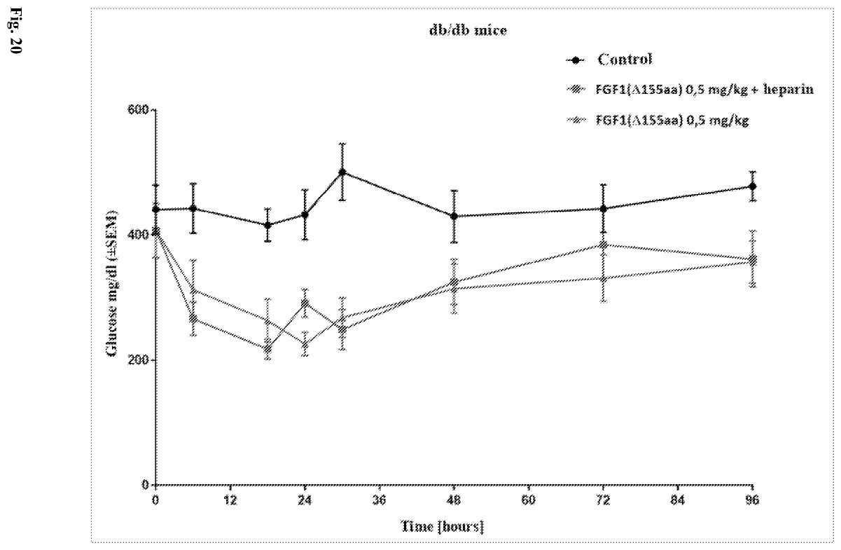

The test as carried out was aimed at confirming the antidiabetic activity of the obtained FGF1(Δ155aa) in accordance with the invention. db/db mice were administered subcutaneously 0.5 mg/kg m.c. of the FGF1 protein (Δ155aa) with an addition of heparin (the unchanged FGF1 protein, that is why heparin was used to protect the protein against degradation and inactivation). The protein effectively reduces glucose concentration up to 30 h after injection. db/db mice which received only a carrier were used as the control group (FIG. 20).

### Example 3

Confirming the effectiveness of FGF1(Δ155aa) and the truncated variant-FGF1(Δ155aa; Q55/S62/H108)—without heparin addition.

The test examined the antidiabetic effectiveness of FGF1(Δ155aa) protein without the of heparin and effectiveness of the truncated variant-presence FGF1(Δ155aa; Q55/S62/H108). Ultimately, proteins without the presence of heparin in the buffer were to be administered, because administration of heparin causes side effects and that is why it cannot be administered to human subjects.

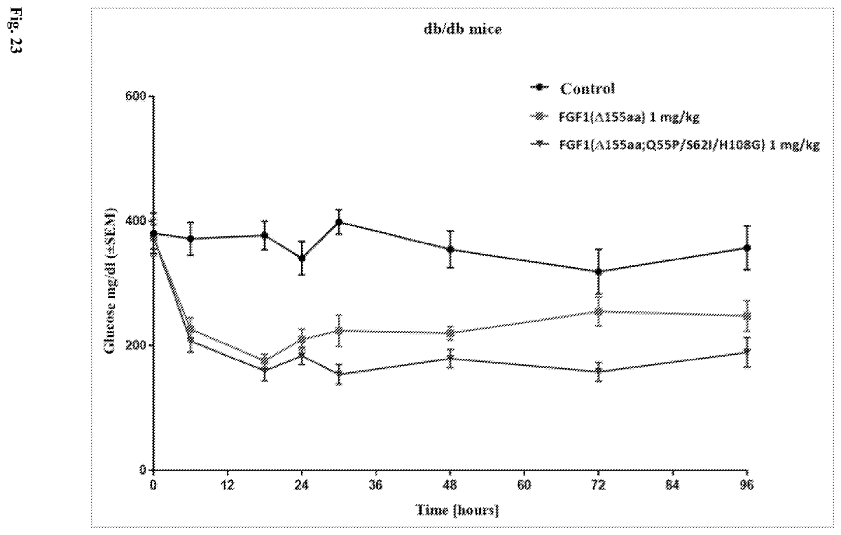

The test was performed d with the use of proteins: FGF1(Δ155aa) and FGF1(Δ155aa; Q55/S62/H108) variant at 1 mg/kg m.c. concentration. Both proteins exhibited antidiabetic activity but the mutein with stabilizing mutations (the stable variant)—FGF1(Δ155aa; Q55/S62/H108)—maintained the effect considerably longer (FIG. 23). Based on the obtained results it was concluded that stabilizing the FGF1 prolongs antidiabetic activity.

By introducing the known stabilizing mutations described in the literature, preferably all such three stabilizing mutations as described above, to the mutated human FGF1 protein according to the invention, a thermally stabilized protein has been obtained which also has a high antidiabetic potential. This also increases the pharmacological usability of this protein, because a stable protein can be efficiently produced on a large scale. However, the mitogenic effect, which is one of the main factors limiting the clinical use of rhFGF1, still has not been eliminated.

### Example 4

Reducing the mitogenic potential of the human FGF1 protein.

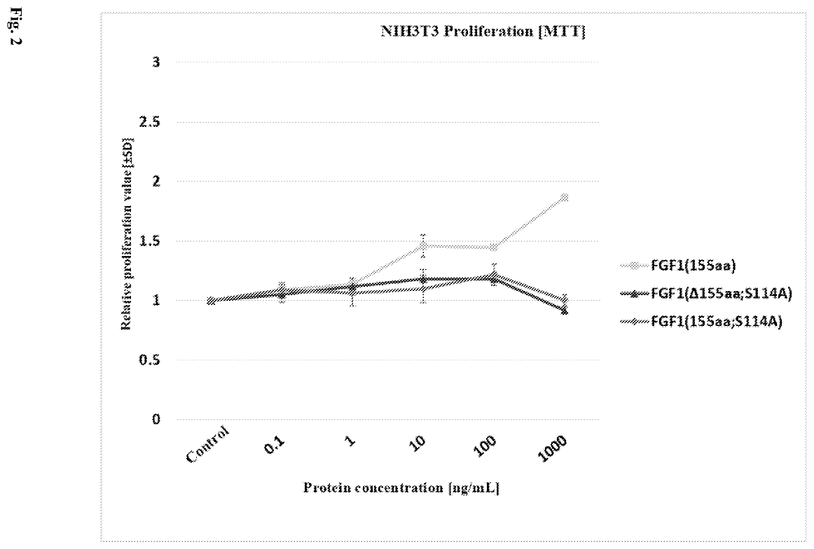

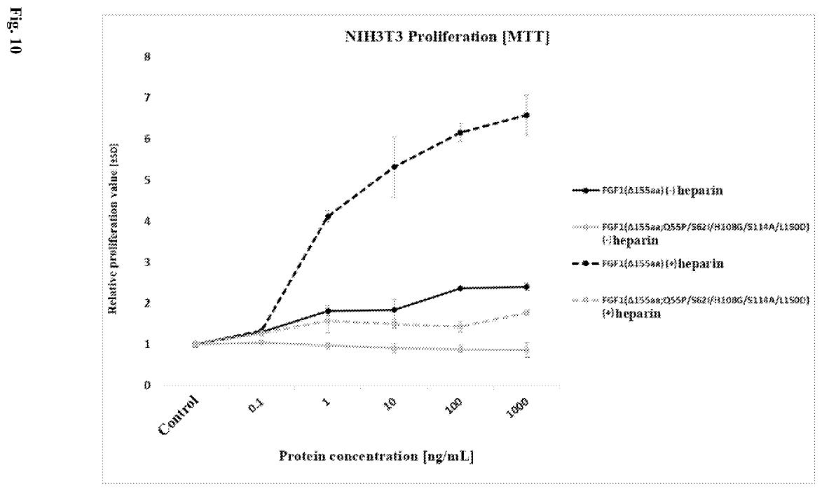

The inventors investigated the mitogenic potential of the truncated human FGF1 protein with introduced S114A mutation in a system without heparin in the cell line NIH 3T3 using a method described by Skjerpen at al. (Skjerpen at al., 2002). In the experiments conducted in the cell line NIH 3T3, the FGF1(Δ155aa; S114A) protein does not stimulate proliferation of cells compared to the unchanged FGF1 (FIG. 2). The results obtained are different from the results obtained by Skjerpen et al. It is necessary to take into account two variables that differentiate both experiments. First, in the study by Skjerpen et al. the experiments were conducted in a system that included also heparin which stabilized the protein. In the Applicant's experiments the effect was observed for the protein which was not additionally stabilized with heparin because protein is administered in vivo without heparin due to its toxic effect on an organism. Moreover, the Applicant demonstrated that the presence of heparin is not necessary to demonstrate the mitogenic potential of the wild-type protein (FIG. 10). Second, Skjerpen et al. worked with a full-length protein whereas the Applicant performed their experiment using both versions: the truncated FGF1(Δ155aa) and the full-length FGF1(155aa).

### Example 5

Impairment of interaction with the FGFR1 receptor by introduction of a mutation in another site than the heparin binding site.

The activation and dimerization of the FGF1 receptor (FGFR1) is necessary to induce mitogenic response. Impairment of ligand binding to the receptor weakens interaction, reduces signal transduction and does not promote cell proliferation.

Variants of FGF-1 were generated which were characterized by impaired binding to the FGFR1 receptor. The mutation was developed within the domain responsible for affinity to the receptor but the modified residue is not crucial for this binding. The Leucine (L) residue at C-terminus was substituted with aspartic acid (D) (L150D).

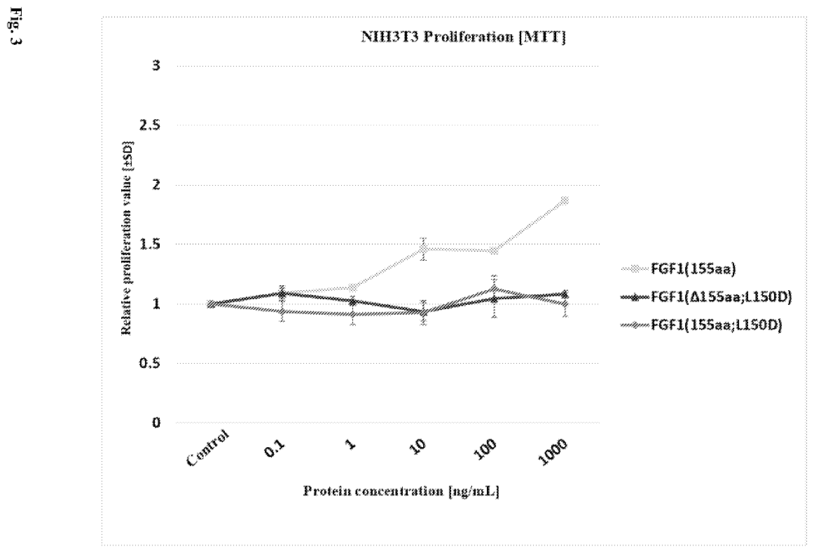

Subsequently, the applicant verified whether a protein obtained in this way, which poorly binds to the receptor, has a weaker mitogenic potential. To this end an experiment was performed with NIH 3T3 cells, which were exposed to the action of the studied proteins for 48 hours. Subsequently, MTT reactant was added to the cells and the level of absorption was measured. Reduced proliferation of cells treated with FGF1(Δ155aa; L150D), FGF1(Δ155aa; L150D) was observed compared to the cells treated with FGF1(155aa) (FIG. 3).

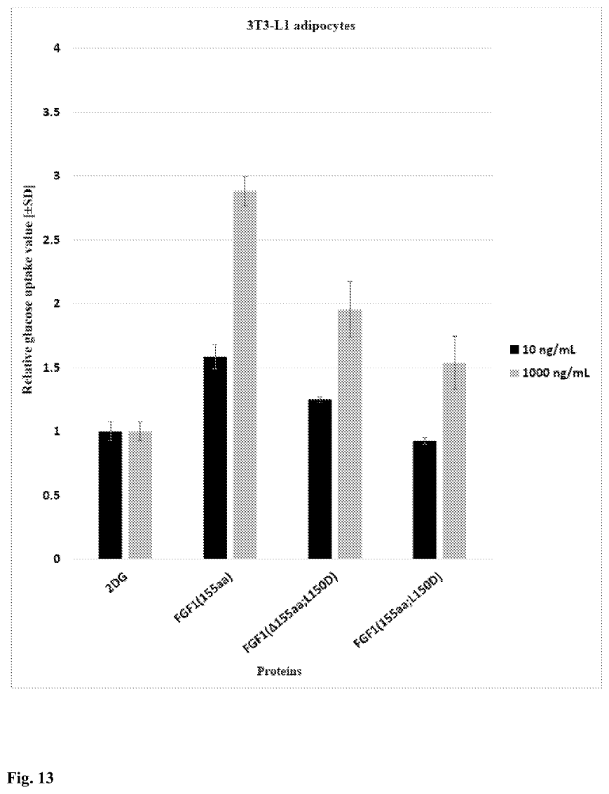

Having obtained the effect of weakened mitogenicity, the inventors verified whether the protein was still characterized by a high antidiabetic activity. In the in vitro assays, 3T3-L1 cells differentiated to adipocytes were treated with the FGF1 protein or a variant: FGF1(Δ155aa; L150D), FGF1(155aa; L150D). After 16 hours from administration, a glucose uptake assay was performed in accordance with the manufacturer's protocol (Glucose Uptake Glo Assay, Promega). In the in vitro assay without addition of heparin the protein induced a slightly increased glucose uptake (FIG. 13).

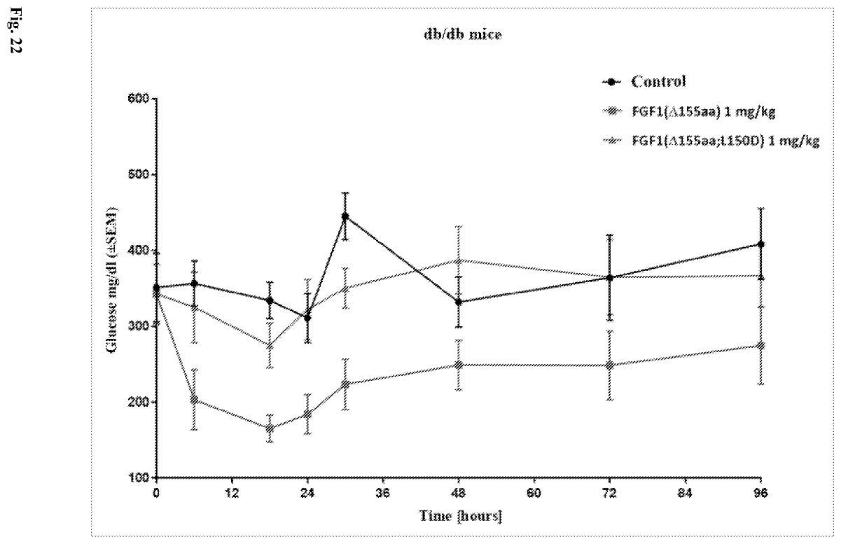

The in vitro results proved to be promising, both in the assessment of mitogenic potential and antidiabetic effectiveness of the new variant-FGF1(Δ155aa; L150D). That is why the new protein with L150D mutation was tested for the ability to reduce blood glucose level in vivo, in the db/db mice animal model. 1 mg/kg dose of the protein was administered subcutaneously to the mice. Subsequently, at several points of time, blood glucose level was measured in the mice by means of a glucometer by puncturing the tip of the tail. Unfortunately, FGF1(Δ155aa; L150D) variant failed to reduce blood glucose in vivo (FIG. 22).

The reason for the lack of effect consists probably in the physical properties of the protein—this mutant is characterized by low stability, its denaturation temperature is about 28° C. Thus, the protein administered subcutaneously is immediately unfolded and heparans naturally present on the surface of cells are not sufficient to stabilize the protein. At the physiological body temperature of mice (about 37° C.) the protein undergoes denaturation and losses its activity.

### Example 6

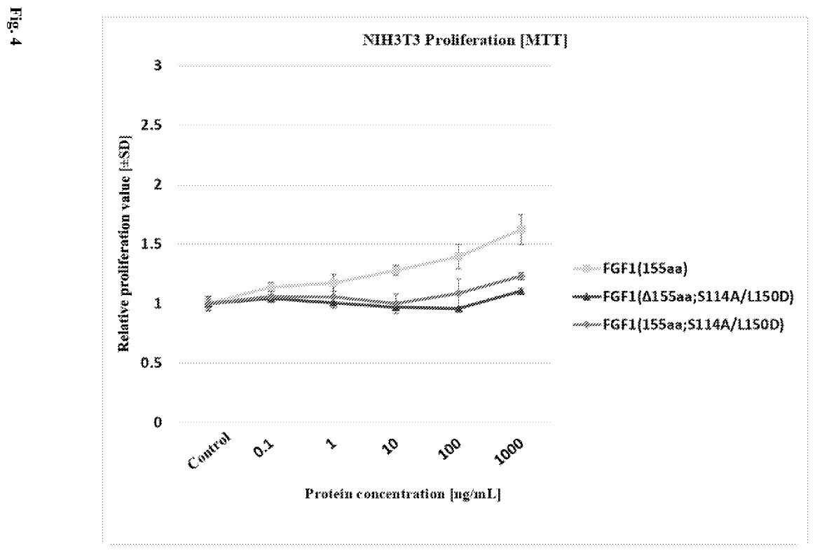

Activity of FGF1 protein variants with double mutation (FGF1(155aa; S114A/L150D)) In the experiment, the activity of a mutant comprising S114A mutation and L150D mutation in NIH 3T3 cells was tested in an MTT assay. It was fund out that such a double mutant exhibits decreased mitogenic properties (FIG. 4). Both S114A mutation and L150D mutation as well as their combination reduce proliferation compared to the wild-type protein. Interestingly, the most decreased mitogenic effect was observed for the stabilized protein, i.e. one additionally comprising the three stabilizing mutations according to the invention FGF1(155aa; Q55P/S62I/H108G), as will be shown below. Additionally, the protein with five mutations—FGF1(155aa; Q55P/S62I/H108G/S114A/L150D)—according to the invention exhibits antidiabetic activity, contrary to the L150D and S114A/L150D variants without stabilizing mutations.

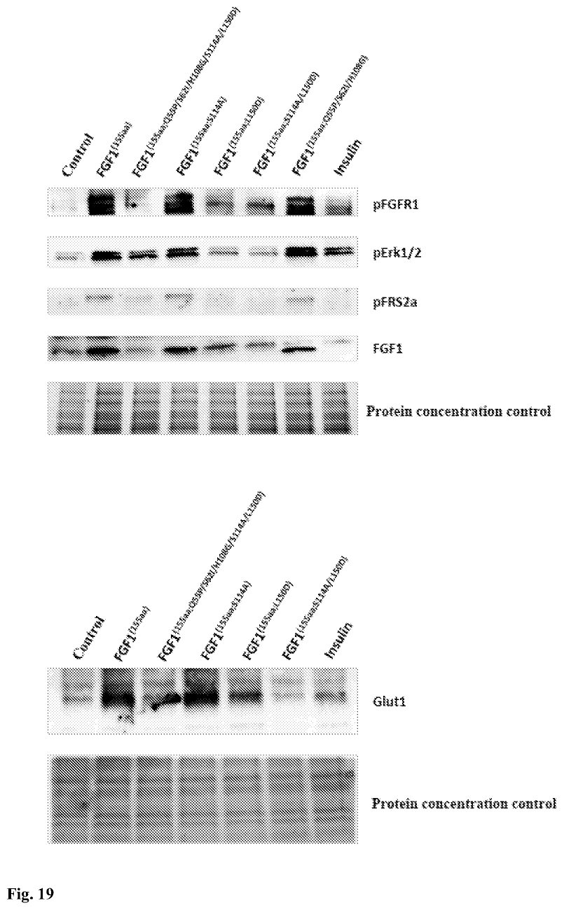

In the experiment testing a signal cascade responsible for triggering a proliferation path, FGF1(Δ155aa; L150D), FGF1(Δ155aa; S114A/L150D) variants are less effective in activating the FGFR1 receptor, which results in a weaker signal transduction and lower phosphorylation of Akt/PKB and ERK kinases (FIG. 19).

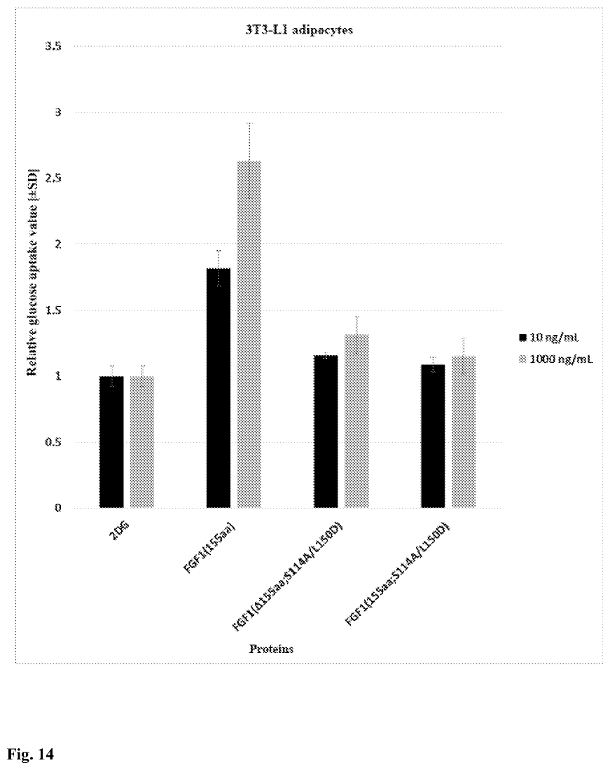

In the glucose uptake assay, a key assay in the context of antidiabetic activity of the FGF-1 protein, FGF1(Δ155aa; L150D), FGF1(Δ155aa; S114A/L150D) variants according to the invention do not induce glucose uptake by mouse adipocytes, even with the highest dose of 1000 ng/ml (FIG. 14). They do not show antidiabetic effectiveness without stabilizing mutations; that is why variants with multiple point mutations were obtained, in particular with five point mutations, both with two mutations reducing mitogenicity (S114A/L150D) and three stabilizing mutations (Q55P/S62I/H108G), which show antidiabetic effectiveness.

### Example 7

Activity of FGF-1 muteins with multiple mutations. FGF1(155aa; Q55P/S62I/H108G/S114A/L150D) and FGF1(Δ155aa; Q55P/S62I/H108G/S114A/L150D) variants

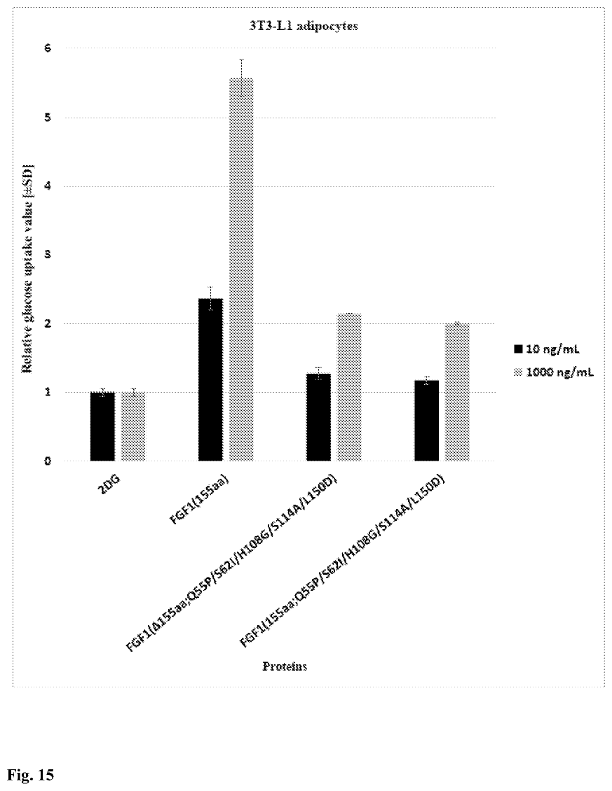

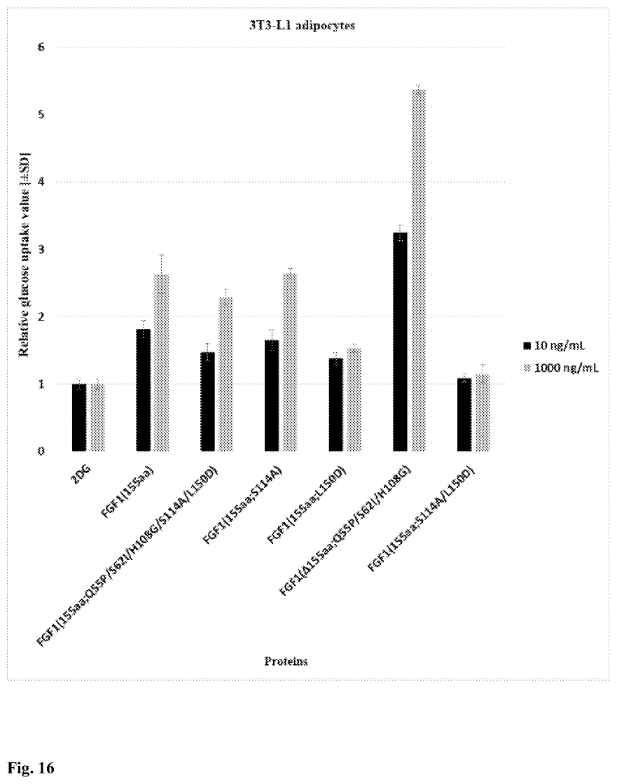

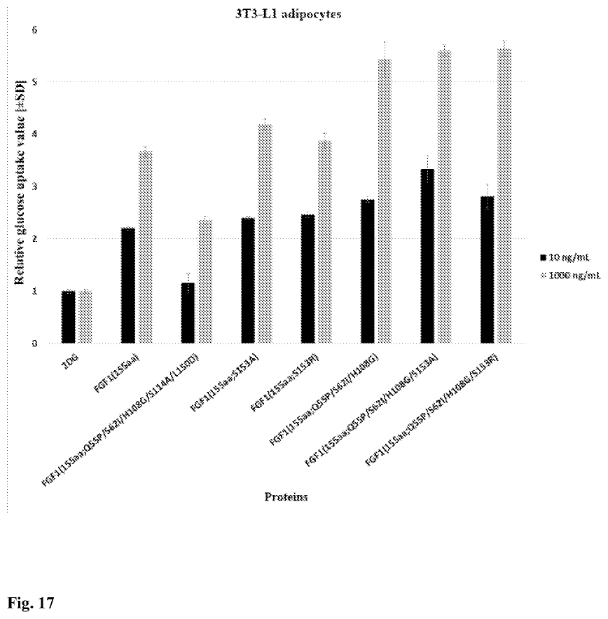

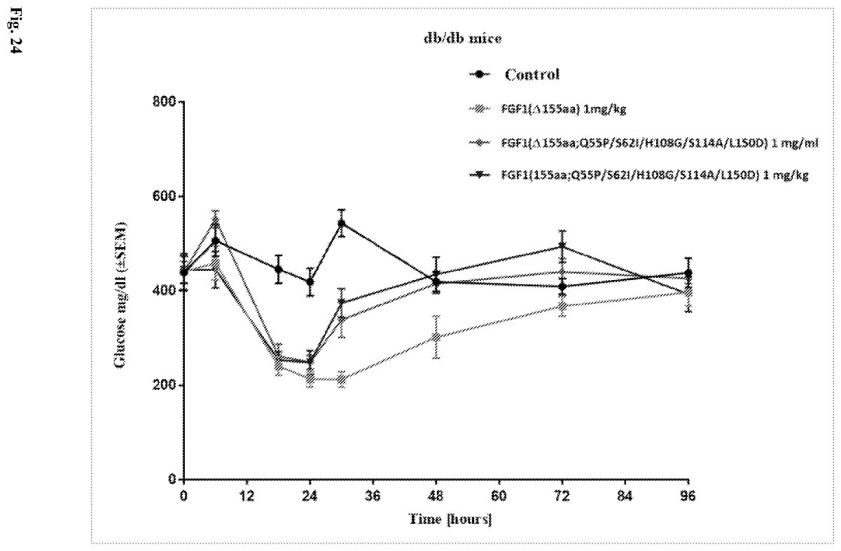

This protein combines all the desired properties: it is stable (denaturation temperature is above 60° C.), it has decreased mitogenicity compared to the wild-type. Wild-type FGF1 protein, even if not stabilized by an addition of heparin to the solution, stimulates proliferation of NIH 3T3 cells. FGF1(155aa; Q55P/S62I/H108G/S114A/L150D) variant at the studied concentrations does not show such an effect, irrespective of the content of heparin or lack thereof (FIG. 10). Moreover, the mutants show antidiabetic activity, both in vitro (in mouse 3T3-L1 cells differentiated to adipocytes) (FIGS. 15, 16, 17) and in vivo. The effect of reduced blood glucose in db/db mice was maintained for the subsequent 30 h after administration of FGF1(Δ155aa; Q55P/S62I/H108G/S114A/L150D) variant and for 48 h after administration of the wild-type protein. Mutated protein is effective, but its activity is shorter than that of the wide-type protein (administered in the same dose to the animals) This results probably from the mechanism of action of FGF1 and the role of FGFR1 receptor in the effect of reducing blood glucose (the mutant has an impaired binding to the FGFR1 due to the presence of L150D mutation). An increase in a dose of mutein to 5 mg/kg m.c. results in achievement of activity time comparable to the wild-type FGF1 administered in a 1 mg/kg m.c. dose. The results of conducted antidiabetic activity assays are presented herein (FIGS. 24,26, 27).

### Example 8

Modifications of the full-length FGF1(155aa; Q55P/S62I/H108G/S114A/L150D) variants FGF1(155aa; Q55P/S62I/H108G/S114A/L150D) variant developed on the full-length FGF1 (155 aa) sequence and the full-length wild-type FGF1(155aa) variant were analyzed to find out whether the 19 aa fragment at N-terminus of the protein has an influence on the biological activity of the protein and its muteins.

The N-terminal deletion of E3-G21 amino acids in the full-length sequence did not change the effect of glucose reduction in vivo (FIG. 24) and the effect was similar to that in the case of analogous full-length muteins (FIG. 15).

### Example 9

Dimeric proteins of the truncated FGF1_DIMER(Δ155aa) variant and the full-length FGF1_DIMER(155aa) variant

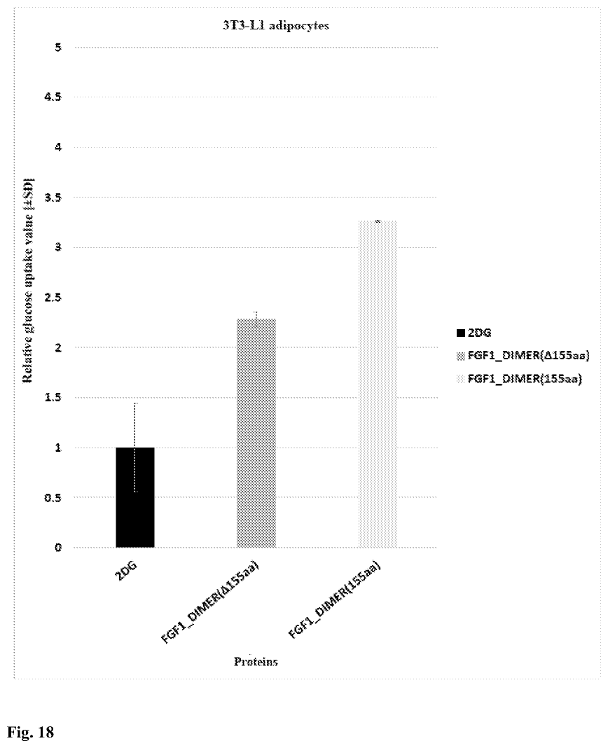

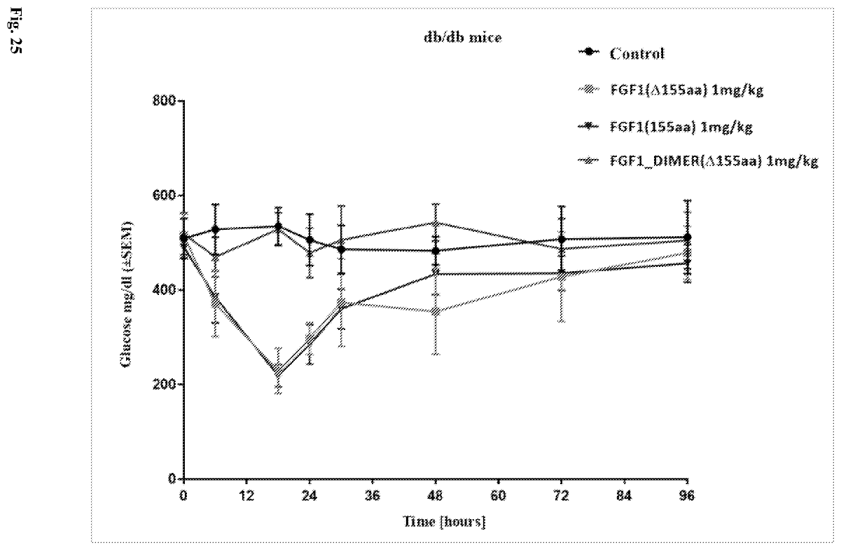

Two molecules of FGF1 were connected by an amino-acid linker in a standard manner. Dimerization was performed in the case of both short and full-length protein sequences. The activity of all these forms is similar in the in vitro conditions (FIG. 18), but in vivo the dimeric FGF1_DIMER(Δ155aa) version was less active (FIG. 25).

### Example 10

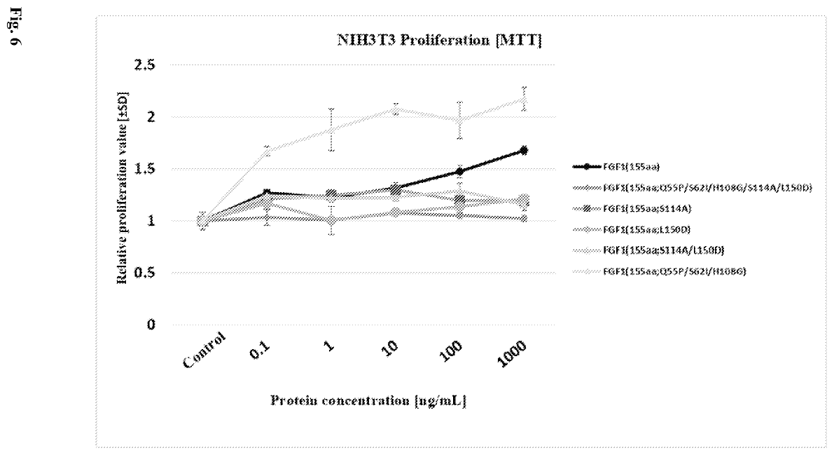

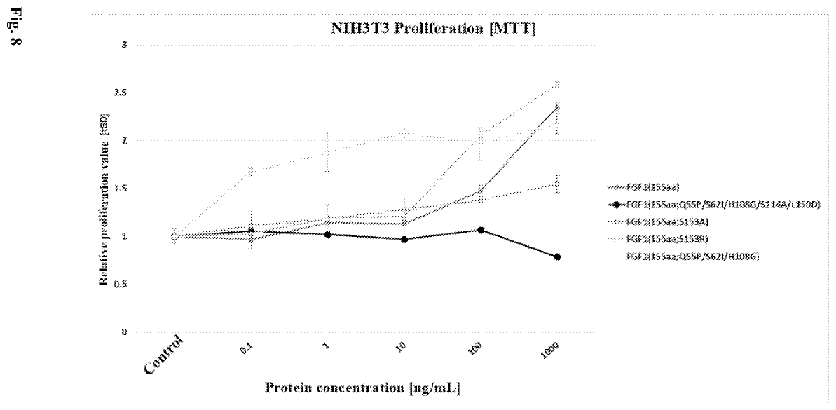

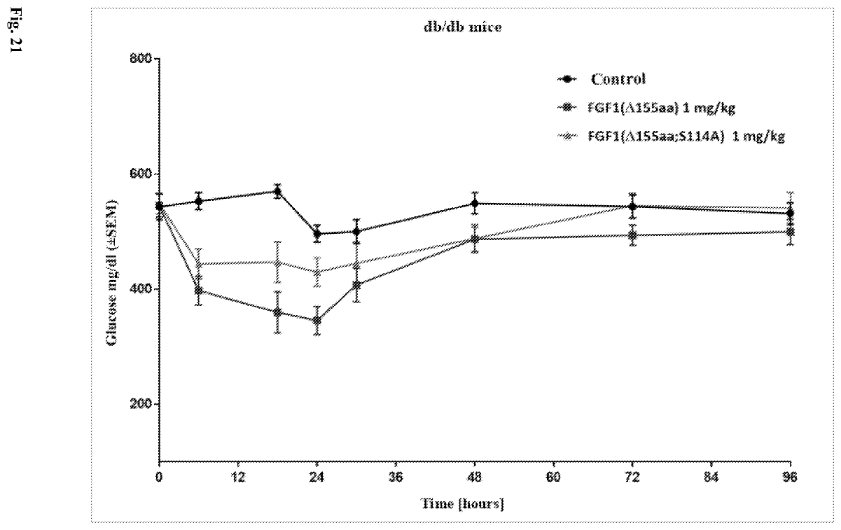

The results showing the activity of the FGF1 protein variants with single mutation: FGF1(155aa; S114A), FGF1(155aa; L150D) and double mutation: FGF1(155aa; S114A/L150D) FGF1(155aa; S114A) and FGF1(155aa; L150D) mutations limit the cell mitogenic activity. The data are presented for NIH 3T3 cells in the MTT assay: both mutation FGF1(155aa; S114A) and FGF1(155aa; L150D) as well as their combination FGF1(155aa; S114A/L150D) reduce proliferation compared to the wild-type protein, i.e. they decrease the mitogenic potential of the mutated protein (FIGS. 2, 3, 4, 6). Interestingly, the most decreased mitogenic effect was observed for the additionally stabilized protein i.e. one additionally comprising the stabilizing mutation according to the invention FGF1(155aa; Q55P/S62I/H108G/S114A/L150D) (FIGS. 6, 7, 8, 10). Additionally, such stabilized FGF1 protein (155aa; Q55P/S62I/H108G/S114A/L150D) exhibits antidiabetic activity, contrary to the variants with single mutations: FGF1(155aa; S114A), FGF1(155aa; L150D) and double mutations FGF1(155aa; S114A/L150D) (FIGS. 21, 22, 24). In the experiment for signal cascade responsible for triggering a proliferation path, FGF1(155aa; S114A), FGF1(155aa; L150D) and FGF1(155aa; S114A/L150D) variants are less effective in activating the FGFR1 receptor, which results in a weaker signal and lower phosphorylation of Akt/PKB and ERK kinases (FIG. 19).

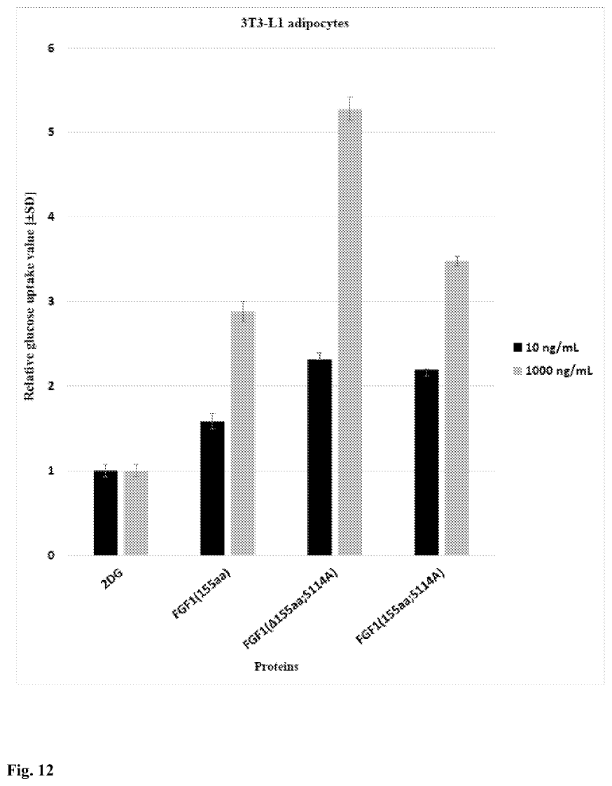

In the glucose uptake assay, a key assay in the context of antidiabetic activity of the FGF1 protein, FGF1(155aa; S114A), FGF1(155aa; L150D) and FGF1(155aa; S114A/L150D) variants according to the invention do not induce glucose uptake by mouse adipocytes, even in the highest dose of 1000 ng/ml (FIGS. 12, 13, 14, 16). They do not show antidiabetic effectiveness without stabilizing mutations; that is why variants with multiple point mutations were obtained, in particular with five point mutations, both with two mutations reducing mitogenicity (S114A/L150D) and three stabilizing mutations (Q55P/S62I/H108G), which, unexpectedly, additionally show antidiabetic activity.

### Example 11

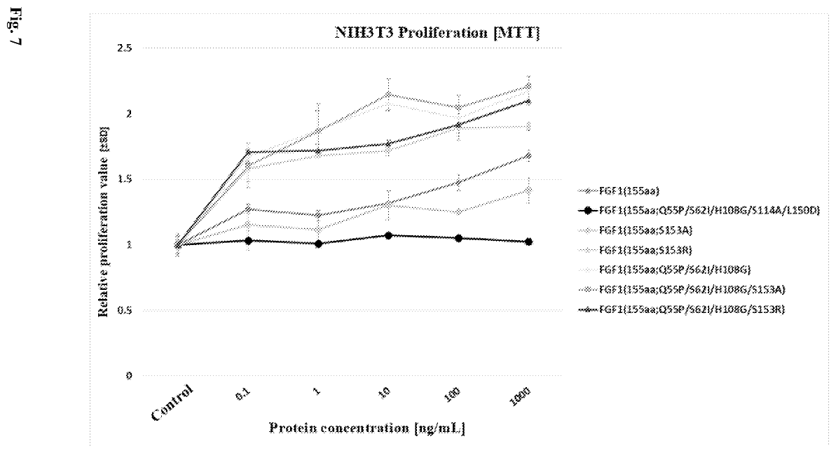

Activity of FGF1 muteins with single mutations at S153 position of the hFGF1 The inventors studied the activity of developed muteins comprising a point mutation located at position S153 in a domain responsible for FGF1 ligand affinity to the receptor, including combinations with the stabilizing mutations according to the disclosure (Q55P, S62I and H108G). The results of studies carried out have showed that the developed muteins at position S153 are characterized by both decreased mitogenicity and the effect of reduced blood glucose level (S153A and S153R-FIGS. 7, 8, 17). Moreover, the multiple variants additionally comprising three stabilizing mutations are characterized by a high thermal stability, resistance to proteolytic degradation and, as a result, a longer half-life in an organism.

1. A human fibroblast growth factor 1 (FGF-1) mutein having decreased mitogenicity, characterized in that it comprises two point mutations: at amino acid position S114 and at amino acid position L150, wherein the numbering of amino acid positions is based on a full-length wild-type FGF-1 protein sequence as shown in Sequence No. 1.

2. The human FGF-1 mutein according to claim 1, characterized in that the point mutation at position S114 is S114A mutation.

3. The human FGF-1 mutein according to claim 1 or 2, characterized in that the point mutation at position L150 is L150D mutation

4. The human FGF-1 mutein according to any one of claims 1 to 3, characterized in that it comprises S114A point mutation and L150D point mutation.

5. The FGF-1 mutein according to any one of claims 1 to 4, characterized in that it further comprises at least one stabilizing mutation at amino acid position selected from: Q55, S62 and H108.

6. The FGF-1 mutein according to claim 5, characterized in that the at least one stabilizing mutation at amino acid position Q55, S62 or H108 is a point mutation selected from Q55P, S62I and H108G, respectively.

7. The FGF-1 mutein according to any one of claims 1 to 6, characterized in that it comprises three stabilizing mutations: Q55P, S62I and H108G.

8. The FGF-1 mutein according to claim 7, characterized in that it has an amino acid sequence presented in Sequence No. 10.

9. The FGF-1 mutein according to any one of claims 1 to 8, characterized in that it additionally comprises N-terminal deletion of at least 19 contiguous amino acids of the full-length FGF-1 protein.

10. The FGF-1 mutein according to claim 9, characterized in that it comprises N-terminal deletion of E3 to G21 amino acids of the full-length FGF-1 protein.

11. The FGF-1 mutein according to any one of claims 1 to 10, characterized in that it comprises S114A point mutation, L150D point mutation and N-terminal deletion of E3 to G21 amino acids of the full-length FGF-1 protein.

12. The FGF-1 mutein according to claim 11, characterized in that it has an amino acid sequence presented in Sequence No. 18.

13. A dimer of human fibroblast growth factor 1 (FGF-1) muteins having decreased mitogenicity as defined in any one of claims 1 to 12.

14. The dimer according to claim 13, characterized in that it is a homodimer.

15. The dimer according to claim 13 or 14, characterized in that muteins forming the dimer are connected by a linker, preferably an amino acid linker.

16. The dimer according to claim 15, characterized in that a linker is an amino acid sequence GGGGGGGGSGGGG.

17. The dimer according to claim 16, characterized in that it has an amino acid sequence presented in Sequence No. 21.

18. The dimer according to claim 16, characterized in that it has an amino acid sequence presented in Sequence No. 22.

19. The human fibroblast growth factor 1 (FGF-1) mutein having decreased mitogenicity as defined in any one of claims 1 to 12 for use in reducing blood glucose level.

20. The human fibroblast growth factor 1 (FGF-1) mutein having decreased mitogenicity as defined in any one of claims 1 to 12 for use in the treatment of diabetes, in particular type 2 diabetes.

21. The FGF-1 mutein for use according to claim 19 or 20, characterized in that it has an amino acid sequence presented in Sequence No. 10.

22. The FGF-1 mutein for use according to claim 19 or 20, characterized in that it has an amino acid sequence presented in Sequence No. 18.

23. The dimer of mutated human fibroblast growth factor 1 (FGF-1) proteins having decreased mitogenicity as defined in any one of claims 13 to 18 for use in reducing blood glucose level.

24. The dimer of mutated human fibroblast growth factor (FGF-1) proteins having decreased mitogenicity as defined in any one of claims 13 to 18 for use in the treatment of diabetes, in particular type 2 diabetes.

25. The dimer of mutated FGF-1 proteins for use according to claim 23 or 24, characterized in that it has an amino acid sequence presented in Sequence No. 21.

26. The dimer of mutated FGF-1 muteins for use according to claim 24 or 25, characterized in that it has an amino acid sequence presented in Sequence No. 22.
